(19) United States
(12) Patent Application Publication
(10) Pub. No.: US 2018/0373272 A1
(43) Pub. Date: Dec. 27, 2018

(54) Fragrance Discharge Device and Control Method Thereof

(71) Applicant: Pium Labs, Inc., Fort Lee, NJ (US)

(72) Inventors: Jaeyeon Kihm, Fort Lee, NJ (US); Sung Jae Hwang, Seongnam-si (KR); Jung Ho Kim, Seoul (KR)

(21) Appl. No.: 16/121,268

(22) Filed: Sep. 4, 2018

Related U.S. Application Data
(63) Continuation-in-part of application No. PCT/KR2017/002274, filed on Feb. 3, 2017.

(30) Foreign Application Priority Data
Mar. 4, 2016 (KR) 10-2016-0026674
Dec. 20, 2016 (KR) 10-2016-0073430

(51) Int. Cl. G05D 7/06; B05B 11/00

(52) U.S. Cl. CPC G05D 7/0629; B05B 11/0037

(57) ABSTRACT

Provided is a fragrance discharge device and control method thereof, the fragrance discharge device including a body configured to be wearable by a user; a storage provided to the body and configured to store a fragrance material; a discharge portion configured to discharge, to an outside of the body, the fragrance material supplied from the storage; and a controller configured to control the discharge portion to selectively discharge the fragrance material based on a result of detecting a motion of the user.

This application is related to concurrently filed U.S. application Ser. No. ______, entitled “Fragrance Discharge Device”, and concurrently filed U.S. application Ser. No. ______, entitled “Mobile Fragrance Discharge Device,” which are herein incorporated by reference in their entirety for all purposes.

### TECHNICAL FIELD

Embodiments relate to a fragrance discharge device and control method thereof.

### BACKGROUND

A fragrance refers to a pleasant odor that may come from flowers, incense, perfume, etc. It has long been known effect that a specific fragrance may affect psychology and emotions of a user. Therefore, people are using perfume for various purposes, such as mood stabilization, psychological stability, and ventilation.

Perfume is generally provided in a liquid form and may be stored and used in a container provided with a device capable of dispensing liquid. In general, perfume in a container is kept at a desired place and used there or by being dispensed by the user over a body part such as a wrist and a neck of the user or clothes of the user, as desired.

However, using such a method, the user needs to move to a place at which the perfume container is placed. For example, the user using a perfume bottle at home may not use perfume while the user is outside and thus, needs to go home to use the perfume.

To improve such convenience, proposed is a device that allows a user to carry and use perfume at an arbitrary place, such as a portable perfume dispensing device disclosed in US Patent Publication No. US2011/0139813 A1. Also, proposed is a device including a fragrance material in a form and a shape aside from liquid and emitting a fragrance, as a dispenser installed in a portable electronic device, disclosed in US Patent Publication No. US2013/0164178 A1.

However, such devices are inconvenient in that they need to be carried by the user separately. In detail, in a situation in which it is difficult for the user to carry a device in either hand, for example, when the user is exercising in simple clothes or performing other tasks with both hands, the user may not readily use the device.

Also, in the case of such devices, the user may need to directly manipulate a device to dispense perfume. Accordingly, the user needs to carry the device to be able to use the device at a desired time to achieve a perfumed effect. If the user forgets to use the device, the effect of fragrance desired by the user may not be acquired. In addition, since the user is required to directly operate the device, the user may not use the device if it is difficult to use the device due to a surrounding environment, for example, when a business meeting is in progress.

### SUMMARY

Embodiments provide a fragrance discharge device that the user may conveniently use and a control method thereof.

Embodiments also provide a fragrance discharge device that may be available even in a situation in which the user may not readily carry with both hands and a control method thereof.

Embodiments also provide a fragrance discharge device that may discharge a fragrance material although a user does not directly manipulate a device and a control method thereof.

According to an aspect, there is provided a fragrance discharge device including a body configured to be wearable by a user; a storage provided to the body and configured to store a fragrance material; a discharge portion configured to discharge, to an outside of the body, the fragrance material supplied from the storage; and a controller configured to control the discharge portion to selectively discharge the fragrance material based on a result of detecting a motion of the user.

Also, the controller may be configured to control the discharge portion by further considering at least one of biometric information of the user, spatial information around the user, a preset time span, a preset space range, and state information of the fragrance discharge device.

Also, at least one of the biometric information, the time information, and the space range may be transferred from an external device.

Also, the fragrance discharge device may further include a sensor portion provided to the body and configured to detect the motion.

Also, the fragrance discharge device may further include a communicator configured to receive a detection result from an external device including a sensor portion configured to detect the motion.

Also, the controller may be configured to transmit at least one of a stored amount of the fragrance material and discharge information of the fragrance material to the external device through the communicator.

Also, the motion of the user may be a single continuous motion, and the controller may be configured to determine whether to discharge the fragrance material in response to a plurality of motions of the user accumulated during a preset period of time.

Also, the body may include a mounter that is detachably provided to at least one of a body of the user and an article worn by the user.

Also, the body may be integrally formed with a wearable device.

Also, the storage may include a plurality of storage spaces configured to independently store a plurality of fragrance materials each emitting a different fragrance, respectively, and the controller may be configured to control one or at least two of the plurality of fragrance materials to be discharged through the discharge portion.

Also, the controller may be configured to change at least one of a type, an amount, a discharge direction, and a discharge pattern of the fragrance material that is discharged through the discharge portion.

Also, the fragrance discharge device may further include an evaporator configured to evaporate the fragrance material stored in the storage or supplied from the storage. The discharge portion may be configured to discharge the fragrance material in a gas state to an outside.

Also, the controller may be configured to modify a discharge condition for discharging the fragrance material based on a result of measuring biometric information of the user and to control the discharge portion to discharge the fragrance material based on the discharge condition.

Also, the fragrance discharge device may further include an artificial intelligence (AI) learner configured to learn a control history of the controller on discharging of the fragrance material based on at least one of the biometric information of the user, the space information around the user, the preset time range, the preset space range, and the state information of the fragrance discharge device.

The controller may be configured to control the discharge portion using a scheme recommended by the AI learner.

Also, the AI learner may be configured to learn the control history using a time series data analysis.

According to an aspect, there is provided a control method of a fragrance discharge device configured to be wearable by a user and to selectively discharge a fragrance material to an outside using a controller, the method including transferring a result of detecting a motion of the user to the controller; determining, by the controller, at least one of a type, an amount, a discharge direction, and a discharge pattern of the fragrance material to be discharged based on the result of detecting the motion of the user; and discharging the fragrance material stored in a storage provided to a body wearable by the user through a discharge portion provided to the body, based on a determination of the controller.

Also, the determining may include determining, by the controller, at least one of the type, the amount, the discharge direction, and the discharge pattern of the fragrance material by further using at least one of biometric information of the user, space information around the user, a preset time range, a preset space range, and state information of the fragrance discharge device.

Also, the fragrance discharge device control method may further include receiving at least one of the biometric information, the time information, and the space range from an external device.

Also, the fragrance discharge device control method may further include detecting the motion of the user using a sensor portion provided to the body.

Also, the fragrance discharge device control method may further include receiving a detection result from an external device including a sensor portion configured to detect the motion, through a communicator.

Also, the fragrance discharge device control method may further include transmitting at least one of a stored amount of the fragrance material and discharge information of the fragrance material to the external device through the communicator.

Also, the fragrance discharge device control method may further include receiving updated information from the external device through the communicator; and adjusting at least one of a type, an amount, a discharge direction, and a discharge pattern of the fragrance material based on the updated information.

A fragrance discharge device and control method thereof according to embodiments may be conveniently used by a user.

Also, the fragrance discharge device may be available even in a situation in which the user may not readily carry with both hands.

Also, the fragrance discharge device may discharge a fragrance material although the user does not directly manipulate a device.

### BRIEF DESCRIPTION OF DRAWINGS

FIG. 1 illustrates an example of an external device to which a fragrance discharge device is mounted according to an embodiment.

FIG. 2 is a block diagram illustrating a configuration of the fragrance discharge device and the external device of FIG. 1.

FIG. 3 is a cross-sectional view illustrating an internal configuration of the fragrance discharge device of FIG. 1.

FIG. 4 is a flowchart illustrating a control method of a fragrance discharge device according to an embodiment.

FIG. 5 is a flowchart illustrating a control method of a fragrance discharge device according to another embodiment.

FIG. 6 is a flowchart illustrating a control method of an external device according to an embodiment.

FIG. 7, (a), (b), (c), and (d), illustrate examples of a user interface screen of an external device according to an embodiment.

FIG. 8 illustrates an example of a fragrance discharge device according to another embodiment.

FIG. 9 is a flowchart illustrating a control method of an external device according to another embodiment.

FIG. 10 illustrates an example of applying the external device control method of FIG. 9.

FIG. 11 is a flowchart illustrating a control method of a fragrance discharge device according to an embodiment.

FIG. 12 is a graph showing an example of applying the fragrance discharge device control method of FIG. 11.

FIG. 13 is a flowchart illustrating a control method of a fragrance discharge device according to another embodiment.

FIG. 14 illustrates an example of applying the fragrance discharge device control method of FIG. 13.

FIG. 15 is a flowchart illustrating a control method of a fragrance discharge device according to another embodiment.

FIG. 16 illustrates an example of applying the fragrance discharge device control method of FIG. 15.

FIG. 17 is a flowchart illustrating a control method of a fragrance discharge device according to another embodiment.

FIG. 18 illustrates an example of applying the fragrance discharge device control method of FIG. 17.

FIG. 19 is a flowchart illustrating a control method of a fragrance discharge device according to another embodiment.

FIG. 20 illustrates an example of applying the fragrance discharge device control method of FIG. 19.

FIG. 21 illustrates an example of a fragrance discharge device according to another embodiment.

FIG. 22 illustrates an example of a fragrance discharge device according to another embodiment.

FIG. 23 illustrates an example of a fragrance discharge device according to another embodiment.

FIG. 24 is a block diagram illustrating a fragrance discharge device and an external device according to another embodiment.

FIG. 25 illustrates a learning process of an artificial intelligence (AI) learner of FIG. 24.

### DETAILED DESCRIPTION

Hereinafter, embodiments will be described in detail with reference to the accompanying drawings such that those having ordinary skill in the art to which the present disclosure pertains may easily practice the present disclosure. The present disclosure may, however, be embodied in many different forms and should not be construed as being limited to the embodiments set forth herein. In addition, in the description of the present disclosure, detailed description of well-known related structures or functions will be omitted when it is deemed that such description will cause ambiguous interpretation of the present disclosure.

The terminology used herein is for the purpose of describing particular embodiments only and is not intended to be limiting. As used herein, the singular forms “a,” “an,” and “the,” are intended to include the plural forms as well, unless the context clearly indicates otherwise.

It will further be understood that the terms “comprises,” “comprising,” “includes,” and/or “including,” when used herein, specify the presence of stated features, integers, steps, operations, components, and/or parts, but do not preclude the presence or addition of one or more other features, integers, steps, operations, components, parts, and/or groups thereof.

Further, the following embodiments are provided for further clear explanation for those of ordinary skill in the art and thus, shapes and sizes of elements shown in the drawings may be exaggerated for the clear explanation.

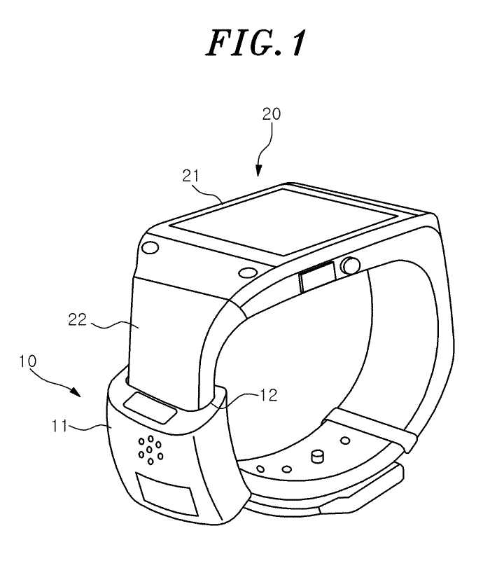

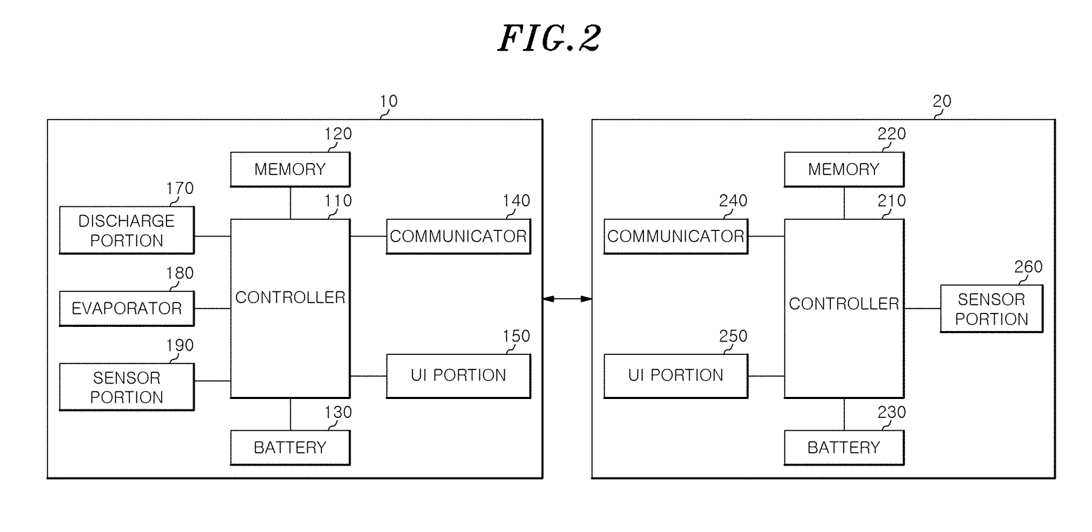

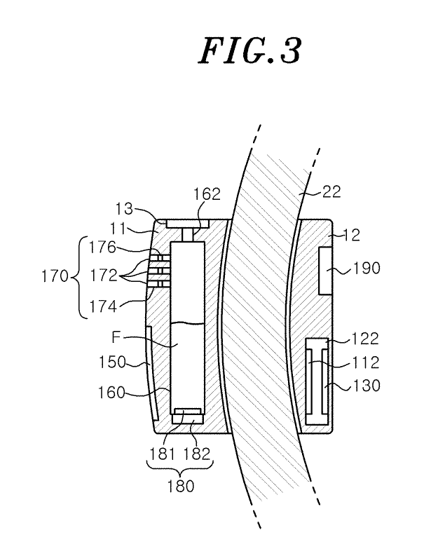

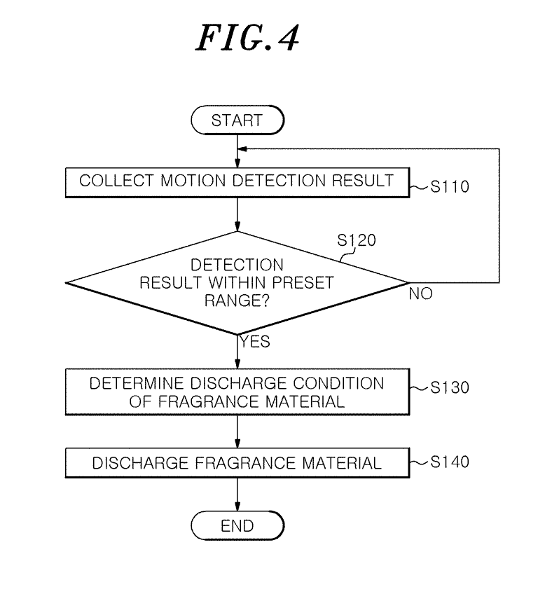

FIG. 1 illustrates an example of an external device to which a fragrance discharge device is mounted according to an embodiment, FIG. 2 is a block diagram illustrating a configuration of the fragrance discharge device and the external device of FIG. 1, FIG. 3 is a cross-sectional view illustrating an internal configuration of the fragrance discharge device of FIG. 1, and FIG. 4 is a flowchart illustrating a control method of a fragrance discharge device according to an embodiment.

Referring to FIGS. 1 through 4, a fragrance discharge device 10 according to an embodiment is configured to discharge a fragrance material stored inside to an outside and may be wearable around a body of a user.

In detail, the fragrance discharge device 10 may include a body 11 configured to be wearable by the user, a storage 160 provided to the body 11 and configured to store the fragrance material, a discharge portion 170 configured to discharge, to an outside of the body 11, the fragrance material supplied from the storage 160, and a controller 110 configured to control the discharge portion 170 to selectively discharge the fragrance material based on a result of detecting a motion of the user. Controller 110 may be any suitable processing device capable of receiving and transmitting digital electronic signals such as, by way of example, a microprocessor, a microcontroller, a digital integrated circuit, etc. Controller 110 may further include or be in communication with a memory 120.

Here, the fragrance material refers to a material capable of emitting a fragrance recognizable through an olfactory sense of a person and a state of a material stored in the storage 160 or a state thereof discharged through the discharge portion 170 is not limited. That is, the fragrance material may be stored in at least one of a liquid state, a gas state, and a solid state in the fragrance discharge device 10 and may be discharged in at least one of the liquid state, the gas state, and the solid state therefrom. Also, the fragrance used herein may also represent a scent and a smell, etc., and thus may be interchangeably used therewith. For example, the fragrance material may be stored in the liquid state in the storage 160 and may be evaporated and then discharged to the outside of the body 11.

Also, the fragrance material may have one or more fragrances. For example, if the fragrance material has a plurality of fragrances, the fragrance material may be a mixture of a plurality of materials each capable of emitting a fragrance.

Also, the fragrance material may have a predetermined function in addition to emitting a fragrance. For example, the fragrance material may be a material with a bacteria removing function, a pain reducing function, or a neutralizing function.

The body 11 may be directly or indirectly wearable around the body of the user, and may have a size and a shape capable of accommodating the storage 160, the discharge portion 170, the controller 110, and the like, and may be formed using a plastic or metal material. Also, although FIG. 1 illustrates that the body 11 is in an approximately rectangular shape, it is provided as an example only. The overall shape of the body 11 may be modified if necessary.

Here, directly wearing the body 11 around the body of the user indicates that directly wearing the body 11 around the body of the user, for example, a wrist, a neck, and a face of the user. Also, indirectly wearing the body 11 around the body of the user indicates that mounting the body 11 around an article worn by the user, for example, a watch, glasses, an accessory, clothes, a bag, and a wearable device. Herein, the expression “the user wears” may be understood to include directly wearing around the body of the user and indirectly wearing by mounting around the article worn by the user.

The body 11 may include a mounter 12 for directly wearing or indirectly wearing as described above. In detail, the mounter 12 may be configured to be suitable for a target to which the body 11 is fastened, that is, a body portion or a type, a shape, a structure, and a location of the article worn around the body of the user. That is, the mounter 12 may be differently configured based on a mounting type of the body 11. For example, the mounter 12 may include a configuration, such as a strap, a ring, a chain, a band, and a belt. Herein, the mounter 12 is provided in a band form in which a strap of a smart watch corresponding to an external device 20 is insertable. Also, the body 11 and the mounter 12 may be integrally formed using the same material. In this case, a boundary between the body 11 and the mounter 12 may be unclear.

In the meantime, the mounter 12 may be configured to be detachable relative to at least one of the body of the user and the article worn by the user. In detail, when the mounter 12 is directly worn around the body of the user, for example, a wrist of the user, the mounter 12 may be configured in a band form. If necessary, the user may wear or take off the fragrance discharge device 10 using the mounter 12. Also, when the mounter 12 is mounted to the article worn by the user, for example, a back sack of the user, the mounter 12 may include a belt and a buckle. The user may wear or take off the fragrance discharge device 10 by adjusting the buckle if necessary. Also, the mounter 12 may be detachably provided to the body of the user and also to the article worn by the user. For this purpose, the mounter 12 may include all of a configuration (e.g., a band) detachable relative to the body of the user and a configuration (e.g., a belt and a buckle) detachable relative to the article worn by the user, and may also include a configuration detachable relative to all of the body of the user and the article worn by the user. For example, when the mounter 12 is a length-adjustable strap, the user may wear the fragrance discharge device 10 around the wrist of the user. If necessary, the user may untie the strap and take off the fragrance discharge device 10 and may adjust the length of the strap and fasten the fragrance discharge device 10 to the back sack. It is provided as an example of the mounter 12. Any known art of detachably providing the body 11 to the body of the user and/or the article worn by the user may be employed for the overall structure, configuration, and mounting method of the mounter 12.

The present embodiment is described based on an example in which the fragrance discharge device 10 is mounted to the external device 20 that is the article worn around the body of the user.

Here, the external device 20 may be a wearable device. Although a smart watch is used as an example of the external device 20, the external device 20 may refer to any device that is wearable around the body of the user. For example, the external device 20 may be various types of devices, such as smart glasses, a smart band, an electronic bracelet, a head mounted device (HMD), electronic clothes, an electronic collar, an electronic accessory, an electronic tattoo, and a smart mirror. The entire configuration of the external device 20 will be further described below.

The storage 160 may store a fragrance material in a predetermined state. Here, the present embodiment is described based on an example in which the fragrance material is stored in a liquid (fluid) state (F) and the storage 160 is in a container form that stores the fragrance material. However, as described above, the fragrance material may be stored in various states and the storage 160 may be in a suitable form corresponding thereto. For example, the fragrance material may be provided in a powder form that includes solid particles. In this case, the storage 160 may include a structure, for example, a membrane, to which powders may be attached and fastened. Also, the fragrance material may be a solidified material emitting a fragrance or a structure including the same. In this case, the storage 160 may be in a structure of fastening the fragrance material to the body 11.

Here, if there is a need to control an environment, such as a temperature condition, for stably storing the fragrance material, the storage 160 may include a device capable of adjusting a storage environment. For example, if there is an optimal storage temperature for improving perfumed effect of the fragrance material, the storage 160 may include a temperature control device, such as a thermoelement.

The storage 160 may provide a storage space capable of storing the fragrance material in a liquid state and may be provided inside the body 11. Although it is illustrated herein as an example that the storage space of the storage 160 is formed only on a side of the body 11, a portion thereof may be formed on a side of the mounter 12.

A supply hole 162 configured to refill the fragrance material in the storage 160 if the fragrance material is out may be formed on one side of the storage 160. Here, a cover 13 may be provided at a location corresponding to the supply hole 162 of the body 11 to selectively block the supply hole 162.

The cover 13 may maintain a fastened state relative to the body 11 using various coupling devices, such as a hook and a bolt, and may be configured to firmly block the supply hole 162 to prevent the fragrance material from leaking through the supply hole 162 although the fragrance discharge device 10 moves strongly by the user. For this purpose, a sealing device such as a gasket may be provided to the cover 13.

In response to initially filling the fragrance material in the fragrance discharge device 10, a decrease in an amount of the fragrance material, or replacing the fragrance material with another fragrance material, the user may open the cover 130 and inject a desired fragrance material into the storage 160 through the supply hole 162. A tool for injecting the fragrance material may be separately provided, such as a dropping pipette.

Also, the fragrance material may fill in the storage 160 with supply of power when charging the fragrance discharge device 10 or the external device 20. For example, a charger (not shown) such as a cradle for charging the fragrance discharge device 10 or the external device 20 may include a storage space capable of storing the fragrance material. A connection device, such as a nozzle, for connecting to the storage 160 of the fragrance discharge device 10 and a supply device, such as an actuator, for supplying the fragrance material may be provided to the charger. In this case, once the fragrance discharge device 10 or the external device 20 is supported over the charger, the connection device provided to the charger may connect to the fragrance discharge device 10 and the fragrance material may be supplied to the fragrance discharge device 10 with charging. In addition to the aforementioned examples, various technical devices may be used as the connection device and the supply device of the charger based on a shape and a structure of the fragrance discharge device 10 or the external device 20. Also, the fragrance discharge device 10 or the external device 20 may transfer a charging signal to the charger and the charger may perform charging only when such a signal is received from the fragrance discharge device 10 or the external device 20.

Also, the storage 160 may be provided in a cartridge form. In this case, the supply hole 162 formed on one side of the storage 160 may serve as a path through which the storage 160 in the cartridge form is led in or led out. The cover 13 may be configured to confine the storage 160 within the body 11. The storage 160 in the cartridge form may be provided to be entirely separate from the body 11 or to be mounted to the body 11 and accordingly, the user may easily replace the storage 160. If a fragrance material emitting a different fragrance is stored for each cartridge, usability may be further enhanced.

Also, the storage 160 may be configured to store a plurality of fragrance materials. In detail, the storage 160 may include a plurality of independent storage spaces and a structure, such as a partition, may be formed in the body 11. Here, a fragrance material having a different fragrance may be stored in each storage space and accordingly, the fragrance discharge device 10 may provide a single fragrance or a plurality of fragrances. In this case, a blending chamber in which a plurality of fragrance materials may mix may be provided on one side of the storage 160. Also, a supply control device capable of selectively adjusting supply of a fragrance material may be provided in each storage space. For example, the storage 160 may include a plurality of storage spaces and a door operating as a solenoid may be formed on a side through which a corresponding fragrance material of each storage is discharged.

The discharge portion 170 may discharge, to an outside, the fragrance material supplied from the storage 160, and may include a discharge hole 172 provided on one side of the body 11 and through which the fragrance material is discharged to the outside.

Here, discharging of the fragrance material may include dispensing the fragrance material to the outside of the fragrance discharge device 10 at predetermined pressure by directly applying a physical external force to the fragrance material and naturally emitting the fragrance material without applying the direct external force to the fragrance material. That is, a discharge method of the fragrance material does not limit the scope of the disclosure. For example, spraying the fragrance material using a predetermined device, naturally evaporating the fragrance material, discharging the fragrance material by force (e.g., inertia) occurring due to a motion of the user may be understood as discharge by the discharge portion 170.

Although it is illustrated herein as an example that a plurality of discharge holes 172 is open toward an outer side of the body 11, it is provided as an example only. A number of discharge holes 172 and an installation direction thereof are not limited thereto. For example, only a single discharge hole 172 may be provided and the discharge hole 172 may be provided on another side of the body 11. Also, the discharge hole 172 may be provided to be open toward another direction of one side of the body 11.

The discharge hole 172 may connect to a discharge passage 174 that extends from the storage 160 and the discharge passage 174 provides a path through which the fragrance material moves to the discharge hole 172. That is, one end of the discharge passage 174 may connect to the storage 160 and another passage thereof may be exposed to the outside of the body 11.

Although it is illustrated herein as an example that the discharge hole 172 directly connects to the storage 160 through the discharge passage 174, a processing portion configured to process the fragrance material using a predetermined method may be further provided between the discharge hole 172 and the storage 160, and the discharge passage 174 may go through the processing portion. For example, the aforementioned blending chamber may be provided to the discharge passage 174 and a pressurization device for increasing the dispensing pressure of the fragrance material or a driving portion 180 may be provided.

A discharge control device 176 configured to control discharging of the fragrance material is provided to the discharge passage 174. Herein, the present embodiment is described based on an example in which a valve capable of adjusting an opening level of the discharge passage 174 is provided as the discharge control device 176. Here, the valve may connect to the controller 110 and may operate with power supplied from the battery 130. In addition, if a plurality of discharge passages 174 is formed as in the present embodiment, the discharge control device 176 may be installed for each discharge passage 174.

Here, the discharge control device 176 may be configured to actively adjust at least one of whether to discharge the fragrance material, a discharge amount, a discharge direction, a discharge pattern, and a type of the fragrance material to be discharged. The discharge pattern may be information including a discharge interval and an amount of the fragrance material to be discharged one time, and a type of the fragrance material. Also, the type of the fragrance material to be discharged may be information including types of fragrance materials to be mixed and a mixing ratio thereof if a plurality of fragrance materials is discharged. Hereinafter, for clarity of description, at least one of whether to discharge the fragrance material, the discharge amount, the discharge direction, and the discharge pattern, and the type of the fragrance material to be discharged is referred to as a “discharge condition”.

Initially, the discharge control device 176 may adjust discharging or non-discharging of the fragrance material and the discharge amount thereof. Here, the discharge control device 176 may operate in response to a control signal provided from the controller 110 and may control discharging or non-discharging and the discharge amount of the fragrance material by adjusting an opening level of the discharge passage 174.

Also, the discharge control device 176 may adjust the discharge direction of the fragrance material. For example, a plurality of discharge holes 172 each open in a different direction may be provided to the body 11, and the discharge control device 176 may be provided to correspond to each discharge hole 172 to adjust the discharge direction of the fragrance material by controlling a corresponding fragrance material to be discharged or not discharged through each discharge hole 172 under control of the controller 110. As another example, a single discharge passage 174 may be diverged and thereby connected to the plurality of discharge holes 172. In this example, the discharge control device 176 may be provided at a divergence point to change the discharge hole 172 through which the fragrance material is discharged.

Here, the discharge direction of the fragrance material may be at least one of a direction toward the body of the user, a direction toward the article worn by the user, and a direction toward a space in which the user is present. The discharge passage 174 and the discharge hole 172 may be formed on the body 11 toward a plurality of directions among the aforementioned directions, and the controller 110 may control the discharge control device 176 to discharge the fragrance material in at least one of the plurality of directions toward which the discharge hole 172 is formed.

Also, the discharge control device 176 may adjust the discharge pattern of the fragrance material. The discharge pattern indicates a combination of the discharge amount of the fragrance material, the discharge direction, the type of the fragrance material, a discharge time, a discharge interval, a number of discharges, and the like. For example, the controller 110 may control the discharge control device 176 to discharge the fragrance material at predetermined time intervals, in detail, based on a pattern of discharging of 1 second, stop of 2 seconds, and discharging of 1 second, and the discharge control device 176 may selectively block the discharge passage 174 in response to the control signal of the controller 110 so that the fragrance material may be discharged based on the predetermined pattern.

Also, the discharge control device 176 may adjust the type of the fragrance material to be discharged. For example, a plurality of fragrance materials may be independently stored in the storage 160 and, in response to the discharge passage 174 connecting to each corresponding storage space, each discharge control device 176 may block the discharge passage 174 so that only a selected fragrance material may be discharged under control of the controller 110. In particular, if the blending chamber is provided between the storage 160 and the discharge hole 172, the discharge control device 176 may adjust a type of a fragrance material that is to be provided to the blending chamber. Accordingly, the fragrance discharge device 10 may provide various fragrances to the user.

The discharge control device 176 may be a device capable of automatically adjusting the discharge condition based on a fragrance material state in addition to acting as the aforementioned active control device. Hereinafter, an example in which cortex capable of selectively allowing only gas to pass is used for the discharge control device 176 will be described. In this case, the discharge control device 176 may be provided to substantially block the discharge passage 174. Since the cortex may allow only gas to pass instead of allowing liquid to pass, the discharge control device 176 may prevent the fragrance material stored in the liquid state in the storage 160 from leaking to the outside. As another example, the discharge control device 176 may be a hole with a small diameter through which liquid may not pass and only gas may pass. That is, the discharge passage 174 itself may function as the discharge control device 176.

The aforementioned examples of the discharge condition are only an embodiment and the discharge condition may be variously modified if necessary. Here, the user may modify the discharge condition using a user interface (UI) portion 150 of the fragrance discharge device 10 or an UI portion 250 of the external device 20.

Also, the discharge condition may be adjusted by another device in addition to the discharge control device 176. For example, in the case of discharging a fragrance material through evaporation, the controller 110 may adjust a discharge amount by adjusting an evaporation amount. Also, the controller 110 may adjust the discharge condition by controlling the driving portion 180.

In detail, the body 11 may further include the driving portion 180 configured to supply energy for discharging the fragrance material. In detail, the driving portion 180 may be supplied with power from the battery 130 or force applied from the user to discharge the fragrance material to the outside of the body 11, and may directly apply, for example, heat, vibration, force, and pressure to the fragrance material or the storage 160 storing the fragrance material as the energy for discharging the fragrance material. To this end, the driving portion 180 may include a predetermined mechanism. For example, the driving portion 180 may be a device capable of directly transferring energy to the fragrance material, such as a piston, a squeezer, a heater, a spray, and a fan, or may be a cylinder device configured to discharge the fragrance material by selectively opening an inlet of the storage 110 in which the fragrance material is compressed at high pressure.

Here, the fragrance material stored in a predetermined state in the storage 160 may be discharged to the outside in the state as is or may be converted from the state to another state and thereby discharged to the outside with the energy supplied through the driving portion 180. For example, when the fragrance material is stored in a liquid state in the storage 160 as in the present embodiment, the fragrance material may be dispensed in the liquid state or may be evaporated and dispensed in a gas state.

The driving portion 180 may be discharged as a single component of the discharge portion 170 and may also be provided separate from the discharge portion 170. Also, the driving portion 180 may operate under control of the controller 110, separate from the discharge portion 170.

The present embodiment is described based on an example in which an evaporator configured to evaporate the fragrance material in the liquid state is provided as the driving portion 180 separate from the discharge portion 170 and independently operates under control of the controller 110. Herein, for clarity of description, reference numeral 180 also refers to the evaporator that is one example of the driving portion 180.

The evaporator 180 may be disposed on one side of the storage 160 and may include a vibrating device 181 configured to evaporate the fragrance material stored in the storage 160 using vibration and an actuator 182 configured to actuate the vibrating device 181. The vibrating device 181 may be provided with being submerged under the fragrance material and may be provided on a side relative to the direction of gravity of the storage 160 so that the evaporation may be smoothly performed although a mounting location of the fragrance discharge device 10 changes. The vibrating device 181 may be a vibrator fastened to a predetermined support, and the actuator 182 may be a motor supplied with the power from the battery 130 to vibrate the vibrator.

The controller 110 may control the evaporator 180 to evaporate the fragrance material stored in the storage 160. The fragrance material evaporated by the evaporator 180 may be discharged to the outside of the body 11 through the discharge portion 170.

Although the present embodiment is described based on an example in which the evaporator 180 is in a direct contact with the fragrance material stored in the storage 160, it is provided as an example only. For example, the evaporator 180 may be provided on the discharge passage 174 and may also be installed in the blending chamber if the blending chamber is provided to the body 11. Alternatively, a separate space for evaporation of the fragrance material may be provided in the body 11, the discharge portion 170 may connect to the separate space, and the evaporator 180 may be installed in the separate space.

A sensor portion 190 configured to detect a motion of the user may be provided to the body 11. The sensor portion 190 may include at least one of a gesture sensor, an acceleration sensor, a gyro sensor, and a global positioning system (GPS) sensor to detect the motion of the user. In detail, if the user wearing the fragrance discharge device 10 moves, the motion of the user may be directly or indirectly transferred to the fragrance discharge device 10 and the sensor portion 10 may detect the motion of the user by sensing a movement of the fragrance discharge device 10.

Also, the sensor portion 190 may include at least one of an air pressure sensor, a magnetic sensor, an acceleration sensor, a grip sensor, a proximity sensor, a color sensor (e.g., a red, green, blue (RGB) sensor), a biosensor, a temperature/humidity sensor, an illuminance sensor, a ultraviolet (UV) sensor, an odor sensor, an E-nose sensor, an electromyography (EMG) sensor, an electroencephalogram (EEG) sensor, an electrocardiogram (ECG) sensor, a photo-plethysmography (PPG) sensor, a blood oxygen saturation level (S_{P}O₂) sensor, an infrared (IR) sensor, an iris sensor, a fingerprint sensor, a geomagnetic sensor, a distance sensor, an ultrasonic sensor, and a pressure sensor. Also, the sensor portion 190 may include a video collection device, such as a camera, and a sound collection device, such as a microphone. Also, the sensor portion 190 may further include a control circuit configured to control one or more sensors.

In addition, the sensor portion 190 may further include a sensor configured to measure an amount of the fragrance material remaining in the storage 160 and a discharge amount of the fragrance material discharged through the discharge portion 170. For example, the sensor portion 190 may further include a weight sensor providable on one side of the storage 160. Here, the remaining amount of the fragrance material may be directly calculated through the weight sensor and the discharge amount thereof may be calculated based on a difference between an initial weight and the remaining amount. Alternatively, the sensor portion 190 may further include a flow amount sensor provided to the discharge portion 170. Here, the discharge amount of the fragrance material may be acquired by accumulating a value measured at the flow amount sensor and the remaining amount thereof may be calculated based on a difference between an initial stored amount and the discharge amount. Also, the sensor portion 190 may include all of the weight sensor and the flow amount sensor.

Discharge information of the fragrance material including the remaining amount and the discharge amount of the fragrance material may be acquired based on an operational state of the driving portion and the evaporator 180. For example, if an evaporation amount is maintained at a preset level at a time of operating the evaporator 180, the discharge amount may be calculated based on operation information of the evaporator 180.

Through the above configuration, the sensor portion 190 may collect biometric information of the user, spatial information around the user, and discharge information of the fragrance material, and may provide the collected information to the controller 110.

Although it is illustrated herein that the sensor portion 190 is provided to the mounter 12 connected to the body 11, it is provided as an example only. A sensor included in the sensor portion 190 may be disposed at a location suitable for its purpose and may be provided to the body 11. Alternatively, a portion of sensors included in the sensor portion 190 may be provided to the body 11 and another portion thereof may be provided to the mounter 12.

The fragrance discharge device 10 may further include a memory 120 configured to store data, the battery 130 configured to supply power, a communicator 140 configured to connect to the external device 20 and to exchange information therewith, and the UI portion 150.

The memory 120 may store a variety of information sensed by the sensor portion 190, information transmitted to and received from the external device 20 through the communicator 140, and data input from the user and data provided to the user through the UI portion 150. For example, the memory 120 may store motion information of the user, biometric information of the user, spatial information around the user, a time span set by the user, a space range set by the user, state information of the fragrance discharge device 10, and discharge information of the fragrance material. Hereinafter, for clarity of description, at least one of the motion information of the user, the biometric information of the user, the spatial information around the user, the time span set by the user, the space range set by the user, the state information of the fragrance discharge device 10, and the discharge information of the fragrance material is referred to as “motion information of the user and the like”.

Here, the motion information of the user may be understood as a single continuous motion of the user wearing the fragrance discharge device 10 and data in which the motion is accumulated during a preset period of time. For example, the motion information of the user may include information, such as a time at which the motion has occurred, a trajectory of the motion, and strength of the motion.

A single continuous motion may indicate that a value measured at the sensor portion 190 may be recognized as a series of action of the user. A criterion for determining the single continuous motion may be preset or may be set by the user. Also, the preset time associated with the motion information of the user may be set by the user.

For example, a single continuous motion of the user may indicate that the user wearing the fragrance discharge device 10 performs a specific action. The action may be recognized by the gesture sensor, the acceleration sensor, the gyro sensor, and the like. In detail, the single continuous motion may be, for example, an action of raising an arm around which the fragrance discharge device 10 is worn, an action of consecutively performing a reciprocating motion several times, or an action of consecutively clapping several times.

Also, the motion of the user accumulated during a preset period of time may refer to data in which identifiable continuous motions are accumulated during a desired period of time, for example, a level of the user using the arm around which the fragrance discharge device 10 is worn during the period of time and a number of times the user performs the reciprocating motion of the arm during the period of time. For example, if the user repeats the reciprocating motion of the arm during the period of time, the user may be determined to be running.

The biometric information of the user may be understood as body information and emotion information of the user that may be directly measured through the sensor portion 190 or acquired by processing information measured through the sensor portion 190. For example, the biometric information may include at least one of pulse, blood pressure, calorie consumption, a sweat level, a body temperature, a respiratory state, an oral state, EMG, ECG, brainwave, fingerprint, iris, a stress index, excitability, fatigue, and smell, and may include body information and emotion information of the user that may be verified based on the measured information.

In addition, the spatial information around the user may be understood as information on an object interactable with the user or the fragrance discharge device 10 in the space and the space itself to which the user belongs, which may be directly measured through the sensor portion 190 or acquired by processing information measured through the sensor portion 190. For example, the spatial information may include at least one of a location of the user, information of the space in which the user is present, types and locations of objects present around the user or an article, distances between the user or the article and the objects present therearound, density, illuminance, temperature, humidity, smell, content of specific gas included in the air, air pressure, and magnetic field. In addition, the spatial information may include a variety of spatial information and object information.

The time span set by the user refers to a time span having a start time and an end time, and may be preset by the user using the fragrance discharge device 10 and the external device 20 or another device such as a PC and may be stored in the memory 120. Information on the time span may be stored in a predetermined server and may be transferred to the fragrance discharge device 10 through the communicator 140 over a wired/wireless network. For example, the preset time span may include at least one of schedule information, time information on an amount of time used for the user to perform a preset action, and information on a specific time set by the user.

Also, the time span may include a time range that is based on using of the fragrance discharge device 10 and the external device 20. For example, the time span may include at least one of information on an amount of time elapsed after using the fragrance discharge device 10 and an amount of time elapsed from a point in time at which the user wears the fragrance discharge device 10 or the external device 20, and may also include a variety of time information.

Herein, in the time span, the start time and the end time may differ from each other and may be identical to each other. That is, the time span may be a specific time. For example, the controller 10 may control the discharge portion 170 so that a fragrance material may be discharged from the fragrance discharge device 10 when the specific time is elapsed since the user wears the external device 20.

The space range set by the user refers to a space range that may be two-dimensionally or three-dimensionally represented, and may be preset by the user using the fragrance discharge device 10 and the external device 20 or another device such as a PC and may be stored in the memory 120. Also, information on the space range may be stored in a predetermined server and may be transferred to the fragrance discharge device 10 through the communicator 140 over a wired/wireless communication network. For example, the preset space range may be at least one of a specific area two-dimensionally or three-dimensionally representable on a map and a spatial area recognizable by the sensor portion 190.

The state information of the fragrance discharge device 10 may include at least one of an amount of time elapsed after wearing the fragrance discharge device 10, a remaining battery amount, a remaining fragrance material amount, a communication connection state with the external device 20, and a manipulation state of the user through the UI portion 150.

In addition, the discharge information of the fragrance material may include at least one of an amount of the fragrance material remaining in the storage 160, a discharge amount of the fragrance material, a number of times the fragrance material is discharged, a discharge direction of the fragrance material, a discharge time of the fragrance material, a type of the discharged fragrance material, a fragrance material discharge pattern, and a fragrance material discharge history.

The aforementioned information may be measured by the sensor portion 190 of the fragrance discharge device 10 or may be stored in the memory 120 of the fragrance discharge device 10. In addition, the aforementioned information may be information that is measured by a sensor portion 260 provided to the external device 20 or stored in a memory 220 and received through the communicator 140.

In the meantime, a discharge condition may be stored in the memory 120. The discharge condition may be preset and stored in the memory 120, and may be modified based on the motion information of the user, the biometric information, the space information around the user, the time range set by the user, the space range set by the user, the state information of the fragrance discharge device 10, the discharge information of the fragrance material, and the like. For example, the discharge condition may be set to dispense a fragrance material in a single specific pattern if the user performs a single specific action, and may also be set to dispense the fragrance material in another single specific pattern if the user performs another single specific action. The aforementioned information may be stored in the memory 120. Also, the discharge condition stored in the memory 120 may be modified by the user controlling the UI portion 150.

All of a replaceable battery and a chargeable battery may be used for the battery 130. If the replaceable battery is used, a cover for replacing the battery 130 may be provided to the body 11 or the mounter 12. If the chargeable battery is used, a connection terminal for charging may be provided to the body 11 or the mounter 12. Also, the battery 130 may be a wirelessly chargeable battery. Here, wireless charging refers to charging the battery 130 without using a terminal exposed to an outside. Various wireless charging technologies may be applied to the fragrance discharge device 10. For example, the battery 130 may be charged using an electromagnetic induction scheme. The electromagnetic induction scheme refers to a scheme of using an electromagnetic induction principle in which, if an electromagnetic field is generated in a power transmitting coil, electricity is induced from a power receiving coil due to effect of the magnetic field. That is, the battery 130 may include an iron core. If the battery 130 is around a wireless charging device with alternating current (AC), charging may be performed by a constantly changing magnetic field. Alternatively, the battery 130 may be wirelessly charged using a magnetic resonance scheme or an electromagnetic scheme. The electromagnetic wave scheme refers to a scheme of receiving, by a receiver, an electromagnetic wave using a rectenna in which an antenna and a rectifier are combined and converting the received electromagnetic wave to power in response to the electromagnetic wave being generated in a transmitter. Also, the electromagnetic resonance scheme refers to technology that allows a transmitting coil to generate a magnetic field vibrating at a resonant frequency so that energy may be concentratively transferred only to a receiving coil designed based on the same resonant frequency. Ampere's law and Faraday's law are commonly applied to the aforementioned electromagnetic induction scheme, electromagnetic wave scheme, and electromagnetic resonance scheme.

Here, wireless charging of the battery 130 may be performed by a separately provided charger and may be performed through the external device 20. In detail, a charging device configured to generate a magnetic field may be provided to the external device 20, so that the aforementioned wireless charging may be performed at a location corresponding to a location at which the fragrance discharge device 10 is mounted.

Also, the battery 130 may be a solar cell. In this case, a light collector, for example, a PN junction semiconductor, capable of collecting sunlight may be provided on an outer surface of the body 11 or the mounter 12. Alternatively, the light collector may be provided to the external device 20 and the battery 130 may be electrically connected to the light collector of the external device 20 through the mounter 12.

The memory 120 and the battery 130 may be disposed in an installation space 122 formed in the mounter 12. Although it is illustrated as an example that the installation space 122 is formed in the mounter 12, the installation space 122 may be formed in the body 11.

Also, a substrate (not shown) on which an integrated chip (IC) serving as the controller 110 may be provided to the installation space 122. A flexible material may be used for the substrate to be easily mountable.

The communicator 140 is provided to the body 11 as a component for wired/wireless communication with the external device 20. If necessary, the communicator 140 may be exposed to the outside of the body 11. Here, the present embodiment is described based on an example in which the communicator 140 is embedded in the body 11 and performs wireless communication with the external device 20. For example, the communicator 140 may be one of communication modules, such as Bluetooth, beacon, near field communication (NFC), radio frequency identification (RFID), ZigBee, ultra wideband (UWB), and Infrared Data Association (IrDA). An antenna for such communication may be further provided in the body 11. The controller 110 may transmit data to the external device 20 through the communicator 140 or may process data received from the external device 20 and may store the data in the memory 120.

A communicable device through the communicator 140 may be another electronic device aside from the external device 20 and having a wireless communication function, such as a PC, a laptop computer, a tablet PC, and a smart card.

In addition, the UI portion 150 is provided to the body 11. The UI portion 150 may include an input device configured to receive data from the user and an output device configured to output data to the user. Here, the input device and the output device may be configured as a single device, such as a touchscreen. For example, the UI portion 150 may include at least one of a decompression button, a touch button, a jog shuttle button, a dial, a wheel scroll, a keypad, a display, a light emitting diode (LED), a touch panel, a touchscreen, a microphone, and a speaker. In addition, known various input/output devices may be used. Herein, the present embodiment is described based on an example in which the touchscreen capable of simultaneously performing input and output is provided on the outer surface of the body 11.

The user may input or modify information used to adjust the discharge condition of the fragrance material through the UI portion 150. For example, the user may change a type, an amount, a direction, a number of discharges, and a pattern of the fragrance material to be discharged by manipulating the UI portion 150. Also, through the UI portion 150, the user may set determination reference values for the aforementioned motion information of the user, biometric information of the user, space information around the user, time range set by the user, space range set by the user, state information of the fragrance discharge device 10, and discharge information of the fragrance material. For example, by manipulating the UI portion 150, the user may set the fragrance material to be discharged if the user constantly performs predetermined strength of exercise during at least a desired period of time.

The controller 110 may control the memory 120, the battery 130, the communicator 140, the UI portion 150, the storage 160, the discharge portion 170, the driving portion/evaporator 180, the sensor portion 190, and the like. In detail, the controller 110 may adjust the discharge condition of the fragrance material based on at least one of information stored in the memory 120, information collected by the sensor portion 190, and information transferred through the communicator 140, or based on information acquired by processing such information. Also, the controller 110 may perform an operation, such as processing of various data.

The external device 20 may include a body 21 and a wearing portion 22 configured to connect to the body 21 and to be wearable around the body of the user. As described above, the external device 20 may be a wearable device. The present embodiment is described based on an example in which the external device 20 is a smart watch.

In detail, components suitable for use purpose of the external device 20 may be provided to the body 21 of the external device 20. For example, a substrate on which a processing device serving as a controller 210 is installed, the memory 220, a battery 230, a communicator 240, the UI portion 250, and the sensor portion 260 may be provided to the body 21.

Here, the communicator 240 may be a communication module capable of performing wired/wireless communication with the communicator 140 of the fragrance discharge device 10 and may further include another communication module for communication with another device, in addition to the fragrance discharge device 10. The controller 210 of the external device 20 may transfer, to the fragrance discharge device 10, information measured by the sensor portion 260 or information acquired by processing the measured information through the communicator 240. Also, the controller 210 may receive state information of the fragrance discharge device 10, discharge information of the fragrance material, etc., from the fragrance discharge device 10 and may store the received information in the memory 220, or may output the information through the UI portion 250, or may transfer the information to the controller 110.

The sensor portion 260 of the external device 20 may collect information measured by the sensor portion 190 of the fragrance discharge device 10, for example, motion information of the user, biometric information of the user, space information around the user, and discharge information of the fragrance material. A type of a detailed sensor included in the sensor portion 260 and a function of the sensor portion 260 may substantially correspond to those of the sensor portion 190 of the fragrance discharge device 10. Accordingly, a further description is omitted here.

Also, similar to the UI portion 150 of the fragrance discharge device 10, the UI portion 250 of the external device 20 may include an input device and an output device. The user may perform the substantially same functionality as that of the UI portion 150 of the fragrance discharge device 10. That is, the UI portion 250 of the external device 20 may be configured such that the user may adjust a variety information for the discharge condition and discharging of the fragrance material. The controller 210 of the external device 20 may transfer information input through the UI portion 250 to the fragrance discharge device 10 through the communicator 240. A type and a function of a detailed device providable through the UI portion 250 may correspond to those of the UI portion 250 of the fragrance discharge device 10. Accordingly, a further description is omitted here.

In addition, similar to the memory 210 of the fragrance discharge device 10, the memory 220 of the external device 20 may store motion information of the user, biometric information of the user, space information around the user, the time range set by the user, the space range set by the user, state information of the fragrance discharge device 10, and discharge information of the fragrance material. Also, the memory 220 of the external device 20 may further store identification information of the fragrance discharge device 10 for communication with the fragrance discharge device 10 mounted to the external device 20. If a plurality of discharge devices 10 is mounted to the external device 20 or worn by the user, identification information of each discharge device 10 may be configured to be unique and thereby stored.

The battery 230 of the external device 20 may have the substantially same technical feature as the battery 130 of the fragrance discharge device 10. A further description is omitted here.

Hereinafter, an operation and effect of the fragrance discharge device 10 constructed as above will be described.

The fragrance discharge device 10 is mounted to the external device 20 worn by the user and selectively discharges a fragrance material based on a result of detecting a motion of the user. Referring to FIG. 1, if the external device 20 is a smart watch worn around a wrist of the user, the fragrance discharge device 10 may be put on a strap of the smart watch.

In detail, in operation S110, the fragrance discharge device 10 may collect motion information of the user. Here, motion information of the user may be collected through the sensor portion 190 provided to the fragrance discharge device 10 or may be collected through the sensor portion 260 of the external device 20 to which the fragrance discharge device 10 is mounted. In the latter case, information collected by the external device 20 is transferred to the fragrance discharge device 10 through the communicator 140.

The controller 110 may store the collected motion information of the user or information acquired by processing the collected motion information of the user, in the memory 120. The stored motion information of the user may be accumulated over time. Here, information stored in the memory 120 may include the motion information of the user, the biometric information of the user, the space information around the user, the time range set by the user, the space range set by the user, the state information of the fragrance discharge device 10, and the discharge information of the fragrance material.

The motion information of the user may be information on a single continuous motion or motions accumulated during a preset period of time. The motion information may be information acquired through at least one of a gesture sensor, an acceleration sensor, a gyro sensor, and a GPS sensor, or may be information acquired by processing the information.

In operation S120, the controller 110 determines whether the collected motion of the user is within a preset range. In detail, the controller 110 may determine whether the collected motion of the user relates to a motion for discharging the fragrance material or a motion that meets a discharge condition of the fragrance material. Data used as a reference for determination of the controller 110 may be stored in the memory 120. For example, a time range in which a motion of the user consecutively occurs at predetermined strength or more may be set as reference data. The controller 110 may determine whether the accumulated motions of the user meet the reference data during a preset period of time.

If the collected motion of the user is determined to be included in the preset range, the controller 110 may determine the discharge condition of the fragrance material in operation S130. Here, the discharge condition may be preset and stored in the memory 120. Here, the discharge condition may be modified in response to the collected motion of the user and may be constant regardless of the motion of the user. The discharge condition determined by the controller 110 may include at least one of the aforementioned discharge amount, discharge direction, discharge pattern, and fragrance material to be discharged.

Once the discharge condition is determined, the controller 110 may control the discharge portion 170 and the driving portion/evaporator 180 to discharge the fragrance material to the outside based on the discharge condition in operation S140. For example, the controller 110 may control the evaporator 180 to evaporate the fragrance material stored in the storage 160 and may control the discharge control device 176 to open the discharge passage 174, so that the fragrance material may be discharged to the outside.

Here, the discharge information of the fragrance material may be stored and managed in the memory 120 and, if necessary, may be transferred to the external device 20. The external device 20 may store the provided discharge information in the memory 220 and may provide the same to the user using a variety of methods through the UI portion 250.

The fragrance discharge device 10 and the control method thereof according to the present embodiment may allow the user to dispense a desired fragrance material over the body of the user or the space around the user at a desired time while wearing the fragrance discharge device 10 around the body of the user.

Also, since the fragrance discharge device 10 may be directly or indirectly worn around the body of the user, the user may perform tasks further readily using both hands.

In addition, since the fragrance discharge device 10 is able to detect a motion of the user and may determine whether to discharge a fragrance material, the user may enable the fragrance material to be dispensed only with a specific motion, instead of directly adjusting the fragrance discharge device 10, when the user desires to dispense the fragrance material. Also, the controller 110 of the fragrance discharge device 10 may determine whether to discharge the fragrance material based on accumulated motions of the user so that the fragrance material may be automatically dispensed. Accordingly, user convenience may be enhanced and product satisfaction may be improved.

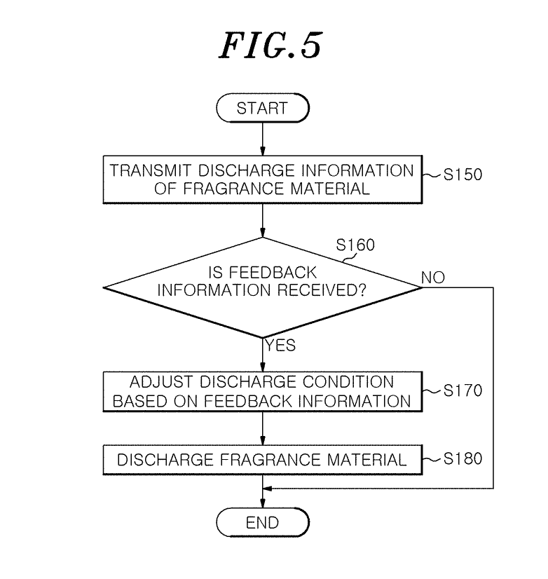

FIG. 5 is a flowchart illustrating a control method of a fragrance discharge device according to another embodiment.

Referring to FIG. 5, in operation S150, the controller 110 of the fragrance discharge device 10 according to another embodiment may transmit discharge information of a fragrance material to the external device 20 when discharging of the fragrance material is completed. Here, as described above, the discharge information of the fragrance material transmitted to the external device 20 may include at least one of an amount of the fragrance material remaining in the storage 160, a discharge amount of the fragrance material, a number of times the fragrance material is discharged, a discharge direction of the fragrance material, a discharge time of the fragrance material, a type of the discharged fragrance material, a fragrance material discharge pattern, and a fragrance material discharge history.

The external device 20 may receive the discharge information from the fragrance discharge device 10 and may collect feedback information of the user or the space around the user over the received discharge information. Here, the feedback information may be motion information of the user, biometric information of the user, and space information around the user newly collected after the fragrance material is discharged from the fragrance discharge device 10. That is, the feedback information may be information updated after discharging the fragrance material. Here, the feedback information may include information collected through the sensor portion 260 and information directly input from the user through the UI portion 250. That is, the user may directly input a feeling of the user or reaction of neighboring persons through the UI portion 250 when the fragrance material is discharged.

The external device 20 may determine whether to transfer the feedback information to the fragrance discharge device 10 based on the collected feedback information. If necessary, the external device 20 may transfer the feedback information as is to the fragrance discharge device 10 through the communicator 240 or may transfer a result of processing the feedback information or a discharge condition modified based on the feedback information. Hereinafter, for clarity of description, the feedback information itself, the result of processing the feedback information, and the discharge condition modified based on the feedback information will be referred to as feedback information.

When the feedback information is transferred from the external device 20 through the communicator 140 in operation S160, the controller 110 may adjust the discharge condition based on the feedback information in operation S170. For this purpose, the controller 110 may store the transferred feedback information in the memory 120 or may perform processing. For example, when a heart rate of the user wearing the fragrance discharge device 10 is determined to be stable based on the feedback information, the controller 110 may modify the discharge condition, such as decreasing an amount of the fragrance material to be discharged and extending a discharge interval.

In operation S180, the controller 110 may discharge the fragrance material based on the a discharge condition.

Although the present embodiment is described herein based on an example in which feedback information is collected by the external device 20 and transferred from the external device 20 to the fragrance discharge device 10, the feedback information may be collected and processed by the fragrance discharge device 10. In detail, when the fragrance material is discharged, the sensor portion 190 of the fragrance discharge device 10 may collect the feedback information, may determine whether there is a need to modify the discharge condition based on the collected feedback information, may modify the discharge condition, and may discharge the fragrance material based on the modified discharge condition.

Also, operations S150 through S180 may be repeatedly performed after discharging the fragrance material.

Also, the fragrance discharge device 10 may suspend discharging of the fragrance material based on the collected feedback information.

Also, a condition for processing and determining the feedback information may be adjusted through the UI portions 150 and 250 of the fragrance discharge device 10 and the external device 20 and thereby stored in the memories 120 and 220.

According to the aforementioned control method of the fragrance discharge device 10 of modifying the discharge condition based on the feedback information, a fragrance material suitable for a preference of the user may be discharged without a need for the user to separately manipulate the fragrance discharge device 10 or the external device 20. Accordingly, user convenience may be enhanced and a user satisfaction for a product may be improved.

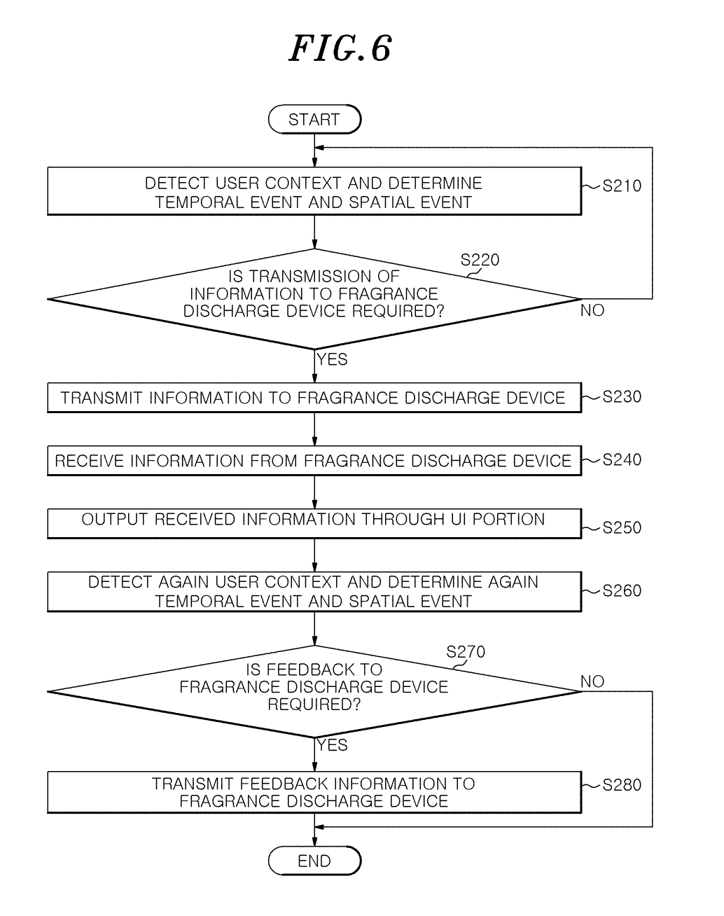

FIG. 6 is a flowchart illustrating a control method of an external device according to an embodiment.

Referring to FIG. 6, in operation S210, the controller 210 of the external device 20 may collect a motion of a user, biometric information of the user, and space information around the user, may determine an occurrence of a temporal event and a spatial event, may collect state information of the fragrance discharge device 10, or may collect discharge information of a fragrance material.

Here, the motion of the user, the biometric information of the user, and the space information around the user may also be referred to as user context as contextual information around the user. The user context may be information measured by the fragrance discharge device 10 and transferred through the communicator 240 or information collected through the sensor portion 260. Also, the temporal event is determined depending on whether a current time is included in a preset time range. If the current time is included in the preset time range, the temporal event may be processed to have occurred. Also, the temporal event is determined depending on whether a current location is included in a preset space range. If the current location is included in the preset space range, the spatial event may be processed to have occurred. Also, the state information of the fragrance discharge device 10 and the discharge information of the fragrance material may be transferred from the fragrance discharge device 10 through the communicator 240. Although the user context and the temporal event and the spatial event are separately described herein, the temporal event and the spatial event may also be referred to as the user context as the contextual information around the user.

In operation S220, the controller 210 may determine whether information associated with discharging of the fragrance material needs to be transferred to the fragrance discharge device 10 based on a result of collecting or determining in operation S210. For example, when the biometric information of the user collected through the sensor portion 260 is determined to indicate an increase in sweat volume or to be greater than a preset value, the controller 210 may determine that the fragrance material needs to be discharged.

When it is determined that the information needs to be transferred to the fragrance discharge device 10, the controller 210 may transfer information associated with discharging of the fragrance material to the fragrance discharge device 10 through the communicator 240 in operation S230. Here, information transferred to the fragrance discharge device 10 may be the aforementioned discharge condition.

The fragrance discharge device 10 may discharge the fragrance material based on the discharge condition transferred from the external device 20 and may transfer state information of the fragrance discharge device 10 or discharge information of the fragrance material to the external device 20.

The external device 20 may receive information transmitted from the fragrance discharge device 10 in operation S240 and may output the received information through the UI portion 250 in operation S250. For example, the external device 20 may receive a remaining amount and a discharge amount of the fragrance material from the fragrance discharge device 10 and may display the received information on a touchscreen.

In operation S260, the controller 210 may collect again the motion of a user, biometric information of the user, and space information around the user, may determine again the occurrence of the temporal event and the spatial event, may collect again state information of the fragrance discharge device 10, or may collect again discharge information of the fragrance material. That is, the controller 210 may collect new information after discharging the fragrance material.

In operation S270, the controller 210 may determine whether a feedback to the fragrance discharge device 10 is required for discharging the fragrance material based on the collected new information. When the feedback is determined to be required, the controller 210 may transmit feedback information to the fragrance discharge device 10 through the communicator 240 in operation S280.

According to the aforementioned control method of the external device 20, the external device 20 may collect information such as a user motion, a configuration of the fragrance discharge device 10 may be simplified. For example, when the fragrance discharge device 10 receives information, such as the user motion, from the external device 20, the sensor portion 190 may be omitted from the fragrance discharge device 10. In this case, it is possible to enhance productivity by reducing production cost of the fragrance discharge device 10 and to further increase storage capacity of the fragrance material. Therefore, user convenience may be further improved.

Also, through an interaction between the fragrance discharge device 10 and the external device 20, the fragrance discharge device 10 may be mounted at further various locations. In detail, the user may wear the external device 20 at a location, for example, a wrist, capable of measuring motion information of the user and may wear the fragrance discharge device 10 at a desired location, for example, a bag, for discharging the fragrance material. In this case, motion information of the user used to determine the discharge condition may include information on a motion of the wrist and the fragrance material may be discharged at a different location. As described above, various scenarios may be configured to discharge the fragrance material through the interaction between the fragrance discharge device 10 and the external device 20.

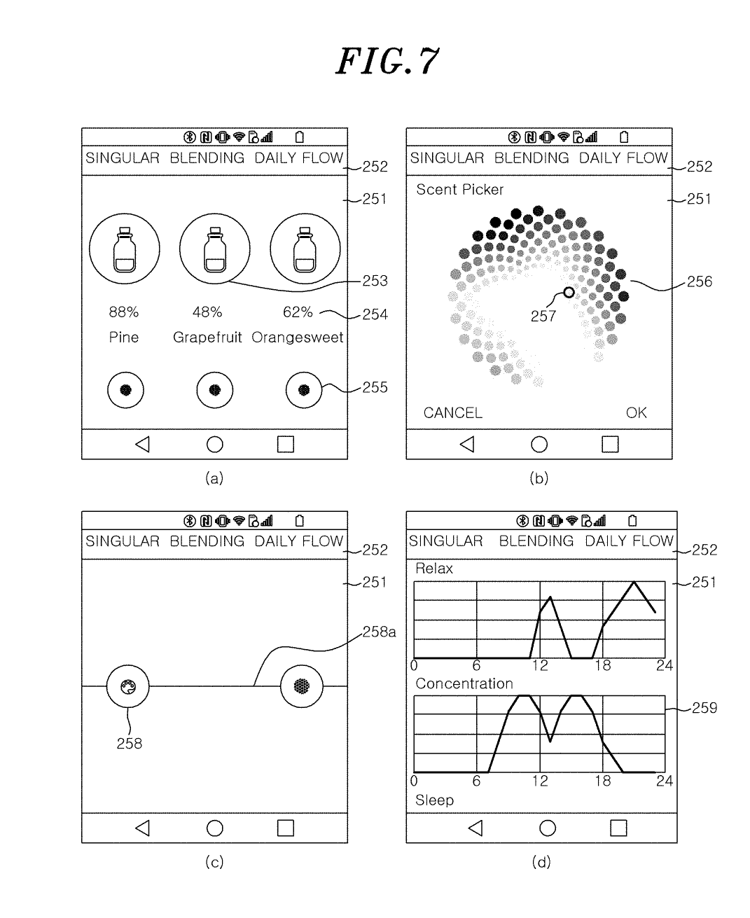

FIG. 7, (a), (b), (c), and (d), illustrate examples of a user interface screen of an external device according to an embodiment.

(a) to (d) of FIG. 7 illustrate examples of an application screen executed on the external device 20. For example, the application screen may be displayed in response to providing a display or a touch screen as the UI portion 250.

In detail, referring to (a) of FIG. 7, a menu area 252 may be displayed on a display screen 251 of the UI portion 250. The user may connect to a desired screen by selecting each menu displayed on the menu area 252. For example, a menu for verifying state information of the fragrance discharge device 10, a menu for verifying discharge information of a fragrance material, a menu for adjusting a discharge condition of a single fragrance material, a menu for setting a method of blending a plurality of fragrance materials and a discharge condition thereof, a menu for adjusting the discharge condition, a menu for verifying a variety of information such as motion information of the user, and a menu for setting a reference value for determining a variety of information such as motion information of the user may be displayed on the menu area 252.

Information 253 and an amount 254 of a fragrance material remaining in the storage 160 may also be displayed on the display screen 251. Herein, three different fragrance materials each emitting a different fragrance (scent) are stored in the storage 160 as an example.

A setting menu 255 for setting a discharge condition of each fragrance material may be additionally displayed on the display screen 251.

Referring to (b) of FIG. 7, the user may conveniently select a desired blending ratio 257 through a mixing ratio selection area 256 on which blending ratios 257 for the plurality of fragrance materials are visually displayed. Shapes/colors/symbols representing mixing ratios of two or more fragrance materials may be visually distinguishably displayed on the mixing ratio selection area 256. The user may touch a shape/color/symbol representing a desired mixing ratio on the mixing ratio selection area 256 and the selected mixing ratio through the touch may be highlighted to be intuitively recognized by the user as indicated by a point 257. The selected mixing ratio may be transferred to the fragrance discharge device 10. The controller 110 of the fragrance discharge device 10 may set the discharge condition based on the transferred mixing ratio and may discharge the fragrance material based on the set discharge condition.

Referring to (c) of FIG. 7, the user may select blending ratios of the plurality of fragrance material through another interface screen. For example, based on a partition line 258a, a surface area of an upper area may represent a relative amount of a first fragrance material and a surface area of a lower area may represent a relative amount of a second fragrance material. The user may adjust a blending ratio of a desired fragrance material by touching and vertically moving the partition line 258a. Also, the user may select a fragrance material to be blended by clicking on a fragrance material selection button 258.

Referring to (d) of FIG. 7, biometric information of the user according to daily schedule may be displayed. The biometric information of the user may be information measured by the sensor portions 190 and 260 of the fragrance discharge device 10 and the external device 20 or may be a value acquired by processing the measured information. Although a stable state and concentration 259 of the user over time is illustrated as an example, a variety of information, such as an amount of time used to take a rest during the day and a state of the rest, an amount of time used to concentrate on a work during the day and strength of the work, and an amount of time used to have a sleep during the day and a state of the sleep, may be displayed. The user may adjust a discharge condition of the fragrance discharge device 10 based on the information and may further conveniently and readily use the fragrance discharge device 10.

Although the present embodiment is described based on an example in which the screen of FIG. 7 is the screen of the external device 20, the present disclosure is not limited thereto. For example, a screen capable of displaying a variety of information displayed on the screens of FIG. 7 may be output through the UI portion 150 of the fragrance discharge device 10 and may also be output through a user interface device of the external device 20 or another device, for example, a smartphone and a TV, communicable with the fragrance discharge device 10.

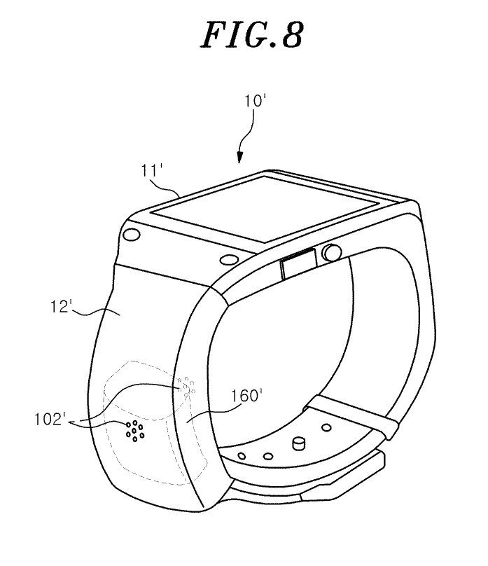

FIG. 8 illustrates an example of a fragrance discharge device according to another embodiment.

Referring to FIG. 8, a fragrance discharge device 10′ according to another embodiment may be a wearable device. In detail, the fragrance discharge device 10′ may include a body 11′ including components for use purpose of the wearable device and a wearing portion 12′ configured to connect to the body 11′ and be wearable around a body of a user. Herein, as an example, the fragrance discharge device 10′ has the functionality of a smart watch.

Here, a storage 160′, a discharge portion 102′, and a driving portion (not shown) may be provided to the body 11′ or a mounter 12′. Although the present embodiment is illustrated as an example that the storage 160′ and the discharge portion 102′ are embedded in a strap of the smart watch, an installation location and a structure may be modified based on a type of the wearable device and a fragrance material discharge condition.

When the fragrance discharge device 10′ is the wearable device as in the present embodiment, the fragrance discharge device 10′ may share and use a sensor portion, a communicator, a UI portion, a memory, and a battery for operating as the wearable device, as a sensor portion, a communicator, a UI portion, a memory, and a battery for discharging a fragrance material.

It may be understood that a storage, a discharge portion, and a driving portion for discharging a fragrance material are added to the wearable device and the controller, the sensor portion, the communicator, the memory, and the battery of the wearable device are used to discharge the fragrance material.

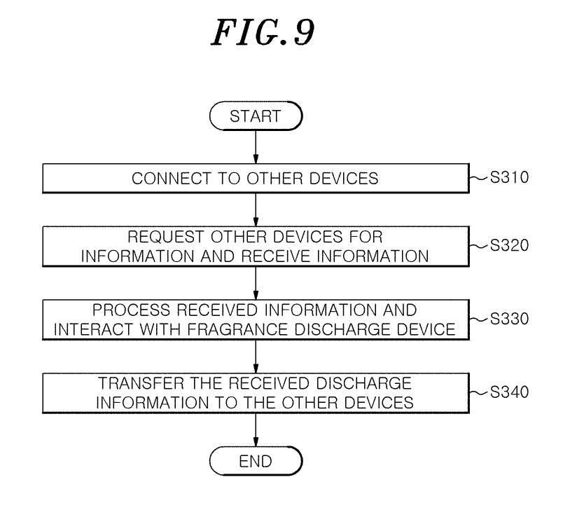

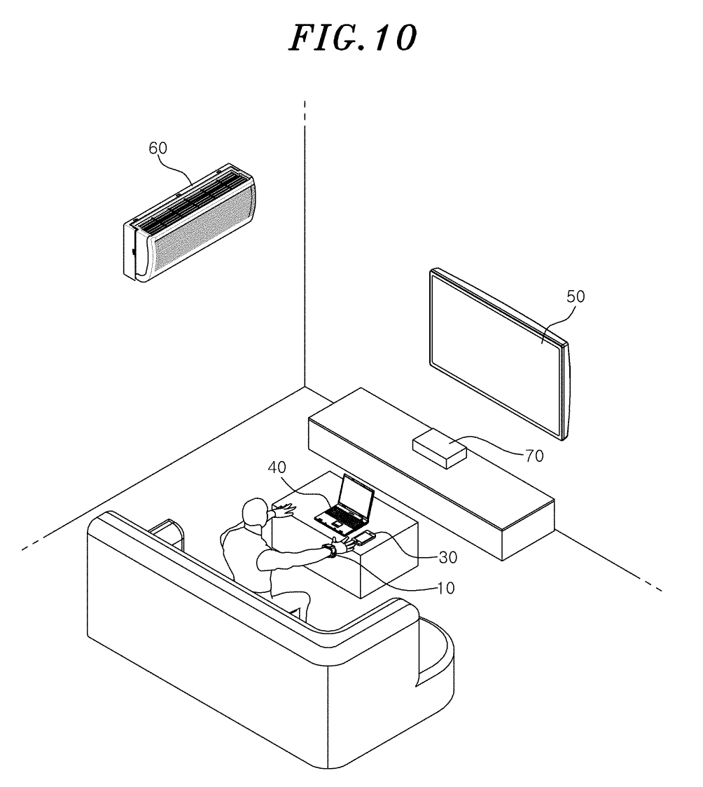

FIG. 9 is a flowchart illustrating a control method of an external device according to another embodiment, and FIG. 10 illustrates an example of applying the external device control method of FIG. 9.

Referring to FIGS. 9 and 10, the external device 20 may interact with other devices in addition to the fragrance discharge device 10. In detail, the other devices may be a smartphone 30, a PC or a laptop computer 40, a TV 50, a smart home device 60 such as an air conditioner, and a virtual reality (VR)/augmented reality (AR) device 70.

The other devices 30, 40, 50, 60, and 70 may include a device configured to collect motion information of the user and a communication device configured to transfer the collected information to the external device 20. For example, a video collection device such as a camera may be provided to the TV 50 and the TV 50 may transfer motion information of the user acquired from the video collection device to the external device 20.

In detail, in operation S310, the controller 210 of the external device 20 may establish a connection for exchanging data with the other devices 30, 40, 50, 60, and 70 through the communicator 240.

In operation S320, the controller 210 may request the other devices 30, 40, 50, 60, and 70 for information used for discharging the fragrance material and may receive the corresponding information. Herein, although the present embodiment is described as an example that the external device 20 requests the other devices 30, 40, 50, 60, and 70, the other devices 30, 40, 50, 60, and 70 may initially transfer the information used for discharging the fragrance material to the external device 20.

In operation S330, the external device 20 may process the information transferred from the other devices 30, 40, 50, 60, and 70, may determine whether the fragrance material needs to be discharged through the fragrance discharge device 10, and when it is determined that the fragrance material needs to be discharged, may interact with the fragrance discharge device 10 so that the fragrance material may be discharged based on the discharge condition.

In operation S340, the external device 20 may receive discharge information of the fragrance material from the fragrance discharge device 10 and may transfer the received discharge information to the other devices 30, 40, 50, 60, and 70. The other devices 30, 40, 50, 60, and 70 may display the information received from the external device 20 for the user through a predetermined output device such as a mounted display.

The aforementioned control method of the external device 20 according to another embodiment may further variously utilize motion information of the user for discharging the fragrance material.

For example, if a condition preset for each device is met, such as when an incoming call is received from a specific user using the smartphone 30, when a specific application is executed on the laptop computer 40, when a specific program is broadcasted on the TV 50, when a preset period of time is elapsed after executing the air conditioner 60, and when a specific scene is displayed on the VR device 70, each of the other devices 30, 40, 50, 60, and 70 may request the fragrance discharge device 10 for discharging a fragrance material corresponding to the preset condition through the external device 20.

Although the present embodiment is described based on an example in which the external device 20 communicates with the other devices 30, 40, 50, 60, and 70 and the fragrance discharge device 10 communicates with the external device 20, the other devices 30, 40, 50, 60, and 70 may directly communicate with the fragrance discharge device 10. For example, the other devices 30, 40, 50, 60, and 70 may directly transfer the collected information and feedback information to the fragrance discharge device 10. In this case, the other devices 30, 40, 50, 60, and 70 may include other features substantially as is, excluding a feature that the external device 20 is worn around the body of the user from among the aforementioned features of the external device 20. In detail, the other devices 30, 40, 50, 60, and 70 may include the controller, the communicator, the sensor portion, the memory, and the battery, and may interact with the fragrance discharge device 10 for discharging the fragrance material, which is similar to the external device 20.

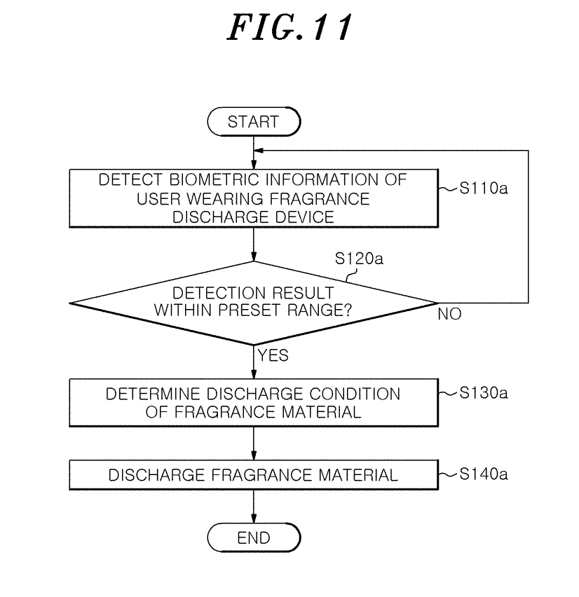

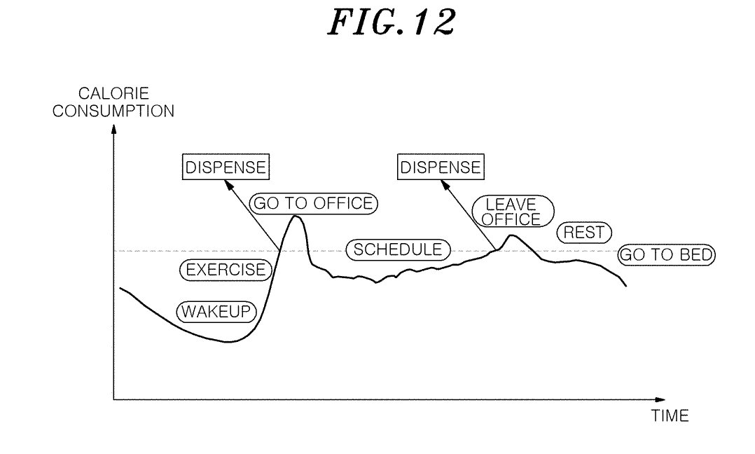

FIG. 11 is a flowchart illustrating a control method of a fragrance discharge device according to an embodiment, and FIG. 12 is a graph showing an example of applying the fragrance discharge device control method of FIG. 11.

Referring to FIGS. 11 and 12, the controller 110 of the fragrance discharge device 10 may discharge a fragrance material based on biometric information of the user.

In detail, in operation S110a, the controller 110 may collect the biometric information of the user by using the sensor portion 190 or by receiving the biometric information of the user collected at the sensor portion 260 of the external device 20 through the communicator 140. For example, the biometric information of the user may be calorie consumption as shown in FIG. 12. The calorie consumption may be measured at preset time intervals, in real time, or in response to a signal generated when the user manipulates the fragrance discharge device 10 or the external device 20.

In operation S120a, the controller 110 may determine whether the collected biometric information of the user is included in a preset range. For example, a reference value for the calorie consumption may be stored in the memory 120 and the controller 120 may determine whether the calorie consumption collected in operation S110a is greater than the reference value.

When the collected biometric information of the user is determined to be included in the preset range, the controller 110 may determine a discharge condition for discharging a fragrance material in operation S130a and may control the discharge portion 170 or the driving portion/evaporator 180 to discharge the fragrance material to the outside based on the discharge condition in operation S140a. Here, the discharge condition may be adjusted to be different based on not only a type of collected information, such as calorie consumption but also different biometric information of the user calculated based on thereon, different collection information, and a collection time. For example, an amount of the fragrance material to be discharged when the biometric information of the user is greater than the reference value may be set to be different in the morning and in the afternoon.

Referring to the graph of FIG. 12, when the user goes to an office or when the user leaves the office, the calorie consumption increases and is greater than a reference value. In response thereto, the fragrance material is dispensed.

Although the present embodiment uses the calorie consumption as an example of the biometric information of the user, the present disclosure is not limited thereto. For example, the biometric information of the user may be an unpleasant odor that may come from the body of the user due to a hormone change. In detail, an odor that comes from the body of the user may vary slightly based on the hormone change, such as physiology and pregnancy of a woman. The sensor portions 190 and 260 may be configured to measure the slightly varying odor. When the user is a female and is determined to be physiological, the controller 110 may modify the discharge condition using various methods, such as increasing a discharge amount or a number of times the fragrance material is discharged, and may discharge the fragrance material based on the modified discharge condition. The discharge condition may be set by the user.

Also, the biometric information of the user may be an index indicating a psychological state of the user. For example, the controller 110 may verify stress, joy, and excitement of the user by complexly considering information such as a heart rate, brainwave, body temperature, pulse, and blood pressure of the user, and may select and discharge a suitable fragrance material. For example, when a stress index of the user is determined to be greater than a preset value, the controller 110 may select and discharge a fragrance material having a function of lowering the stress.

Also, the biometric information of the user may be information acquired by processing information collected at the sensor portions 190 and 260 based on information on a health state of the user, for example, a previous disease history, allergy, high fever, skin trouble, and a heart disease. The information may be pre-stored in the memories 120 and 220 or may be transferred from an external server communicable with the fragrance discharge device 10 or the external device 20.

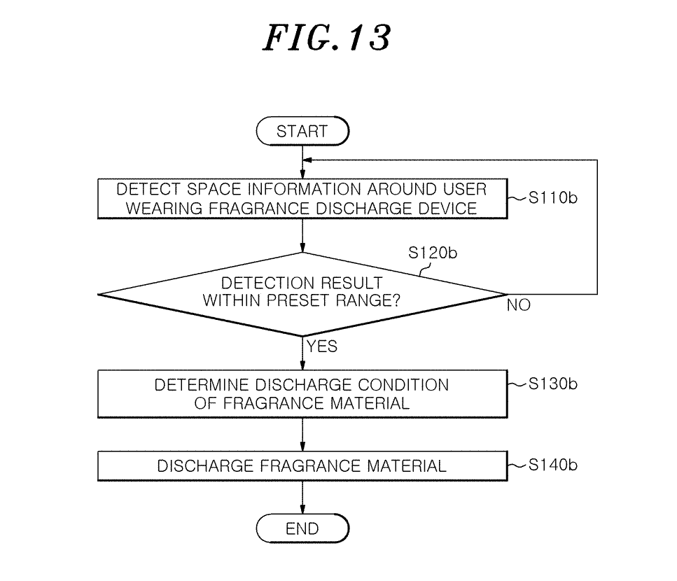

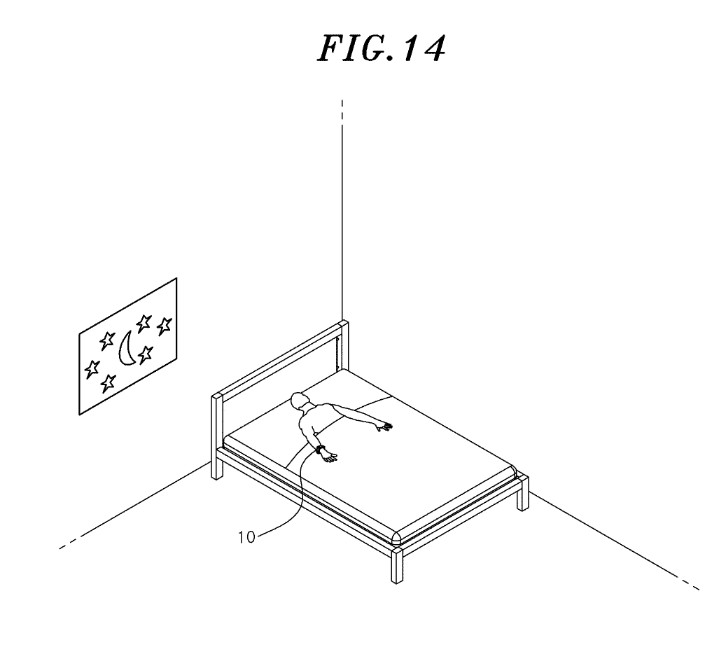

FIG. 13 is a flowchart illustrating a control method of a fragrance discharge device according to another embodiment, and FIG. 14 illustrates an example of applying the fragrance discharge device control method of FIG. 13.

Referring to FIGS. 13 and 14, the controller 110 of the fragrance discharge device 10 may discharge a fragrance material based on space information around the user.

In detail, in operation S110b, the controller 110 may collect space information around the user by using the sensor portion 190 or by receiving the space information around the user collected at the sensor portion 260 of the external device 20 through the communicator 140. For example, the space information around the user may be illuminance that is brightness of light. The illuminance may be measured at preset time intervals, in real time, or in response to a signal generated when the user manipulates the fragrance discharge device 10 or the external device 20.

In operation S120b, the controller 110 may determine whether space information around the user is included in a preset range. For example, a reference value for the illuminance may be stored in the memory 120 and the controller 120 may determine whether the illuminance collected in operation S110b is less than or equal to the reference value. Here, the controller 120 may also consider a temporal element. For example, the controller 120 may determine whether the illuminance is maintained to be less than the reference value during a preset period of time or more.

When the collected space information around the user is determined to be included in the preset range, the controller 110 may determine a discharge condition for discharging a fragrance material in operation S130b and may control the discharge portion 170 and the driving portion/evaporator 180 to discharge the fragrance material based on the discharge condition in operation S140b. Here, the discharge condition may be adjusted to be different based on not only a type of collected information, for example, the illuminance, but also different space information around the user calculated based on thereon, different collection information, and a different collection time. For example, although the same illuminance condition is applied, the discharge condition may be adjusted to be different based on a motion of the user.

FIG. 14 illustrates an example in which the fragrance material is dispensed when the illuminance is maintained to be less than the reference value during a preset period of time or more while the user is lying down on the bed to have a sleep. Here, the fragrance material to be discharged may have a sleep induction function.

Although the present embodiment uses the illuminance as an example of the space information around the user, the present disclosure is not limited thereto. For example, the space information around the user may be density with neighboring persons. In detail, the sensor portions 190 and 260 may verify a location of a neighboring object, for example, a person, using an ultrasonic sensor, a distance sensor, a radar sensor, and a lidar sensor. When a distance therefrom is less than or equal to a present distance or when a number of objects is greater than or equal to a reference number, the sensor portions 190 and 260 may determine that the density is high and may increase an amount of a fragrance material to be discharge or may adjust a direction in which the fragrance material is to be discharged toward the body of the user.

Also, the space information around the user may be a type of a surrounding space. For example, whether the space around the user is a downtown or a forest may be determined based on noise around the space and quality of air. When the space around the user is the forest, the controller 110 may control the fragrance material not to be discharged. Also, the discharge condition may be modified so that a specific fragrance material may be discharged based on the discharge condition.

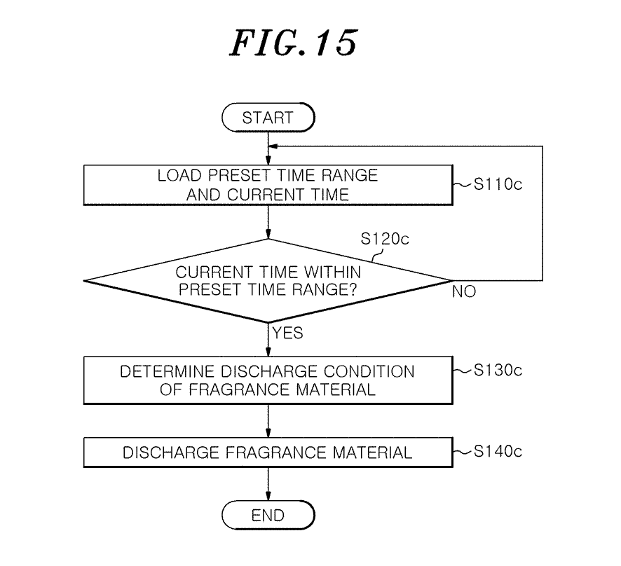

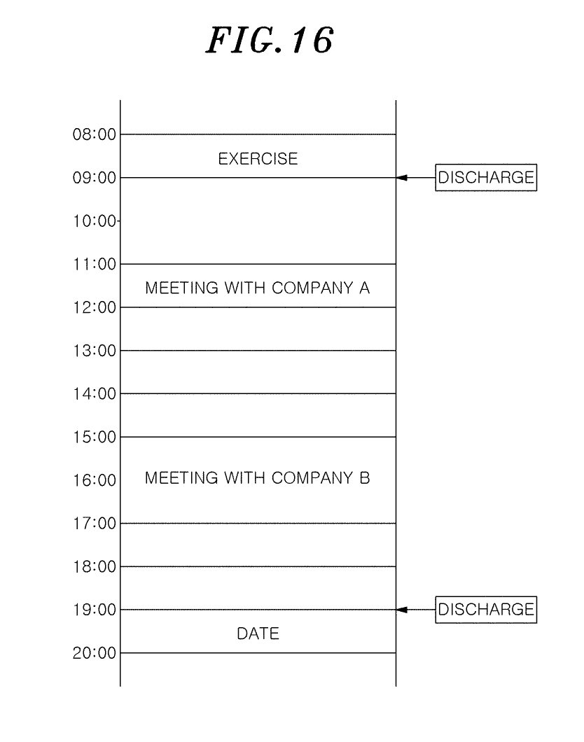

FIG. 15 is a flowchart illustrating a control method of a fragrance discharge device according to another embodiment, and FIG. 16 illustrates an example of applying the fragrance discharge device control method of FIG. 15.

Referring to FIGS. 15 and 16, the controller 110 of the fragrance discharge device 10 may discharge a fragrance material based on a time range set by a user.

In detail, in operation S110c, the controller 110 may load a preset time range stored in the memory 120 or a preset time range transferred from the external device 20 through the communicator 140 and a current time. Here, the preset time range may be the aforementioned specific time. Also, the preset time range may include discharge condition information including information regarding whether to discharge a fragrance material. For example, the preset time range is based on schedule information as shown in FIG. 16, and may include data indicating discharging a fragrance material when exercise ends and when a date starts.

In operation S120c, the controller 110 may determine whether the current time is included in the preset time range. For example, the controller 110 may determine whether the current time is in a range corresponding to 9 o'clock at which exercise ends or whether the current time is in a range corresponding to 19 o'clock at which the date starts, which is a discharge condition of the fragrance material.

When the current time is determined to be included in the preset range, the controller 110 may determine the discharge condition for discharging the fragrance material in operation S130c and may control the discharge portion 170 and the driving portion/evaporator 180 to discharge the fragrance material to the outside based on the discharge condition in operation S140c. Here, the discharge condition may be adjusted to be different depending on whether the current time is included in the preset time range and may also be adjusted to be different based on other collection information. For example, the fragrance material may be set to be discharged when the current time is before 9 o'clock and when calorie consumption of the user is greater than or equal to a preset reference.

FIG. 16 illustrates a daily schedule of the user. Referring to FIG. 16, the user may edit schedule information using the UI portions 150 and 250, and the fragrance material is set to be dispensed in a specific time range, for example, at 9 o'clock at a time when the exercise ends and at 19 o'clock at a time which the date starts. Here, the user may set different fragrance materials to be dispensed at 9 o'clock and 19 o'clock.

Although the present embodiment uses the schedule information as an example of the preset time range, the present disclosure is not limited thereto. For example, the preset time range may be associated with body information of the user. In detail, the preset time range may be physiological information of the user that varies in a predetermined pattern or cycle such as a physiological cycle of a female.

In addition, the preset time range may be pre-stored in the memories 120 and 220 or may be received from an external server communicable with the fragrance discharge device 10 or the external device 20.

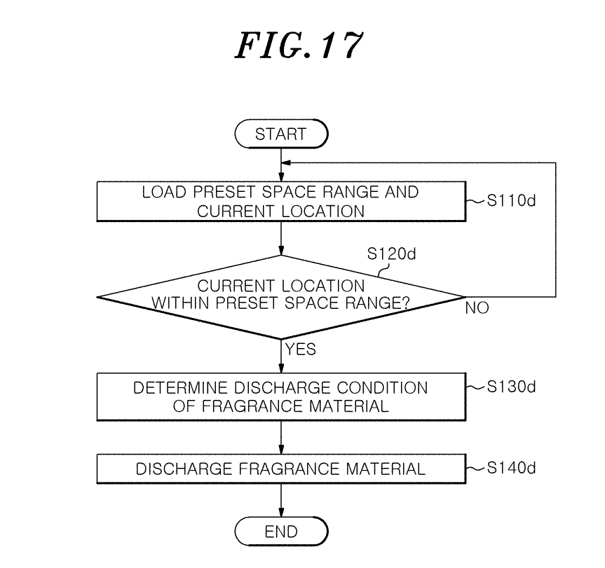

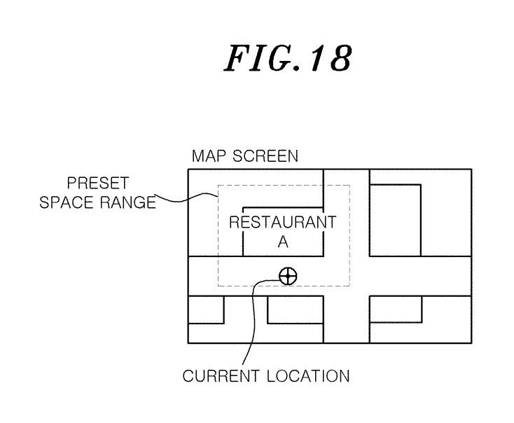

FIG. 17 is a flowchart illustrating a control method of a fragrance discharge device according to another embodiment, and FIG. 18 illustrates an example of applying the fragrance discharge device control method of FIG. 17.

Referring to FIGS. 17 and 18, the controller 110 of the fragrance discharge device 10 may discharge a fragrance material based on a space range preset by the user.

In detail, in operation S110d, the controller 110 may load a preset space range stored in the memory 120 or a preset space range transferred from the external device 20 through the communicator 140 and a current location. Here, the preset space range may be the aforementioned specific location. Also, the preset space range may include discharge condition information regarding whether to discharge a fragrance material. For example, the preset space range is based on two-dimensional (2D) map information as shown in FIG. 18, and may include data indicating discharging a fragrance material when the current location of the user is present within a specific area.

In operation S120d, the controller 110 may determine whether the current location of the user is present within the preset space range. For example, the controller 110 may determine whether the current location is within a specific area that is a discharge condition of the fragrance material.

When the current location is determined to be included in the preset range, the controller 110 may determine the discharge condition for discharging the fragrance material in operation S130d and may control the discharge portion 170 and the driving portion/evaporator 180 to discharge the fragrance material to the outside based on the discharge condition in operation S140d. Here, the discharge condition may be adjusted to be different depending on whether the current location is included in the preset space range and may also be adjusted to be different based on other collection information. For example, the fragrance material may be set to be discharged when the current location is within the specific area and 19 o'clock date is ahead in the schedule information.

FIG. 18 illustrates an example of the preset space range displayed on a 2D map. The user may edit the space range and may set the discharge condition using the UI portions 150 and 250.

Although the present embodiment uses the 2D map as an example of the preset space range, the present disclosure is not limited thereto. For example, a location within a 3D space may be set as the preset space range.

In addition, the preset space range may be pre-stored in the memory 120, 220 or may be transferred from an external device communicable with the fragrance discharge device 10 or the external device 20.

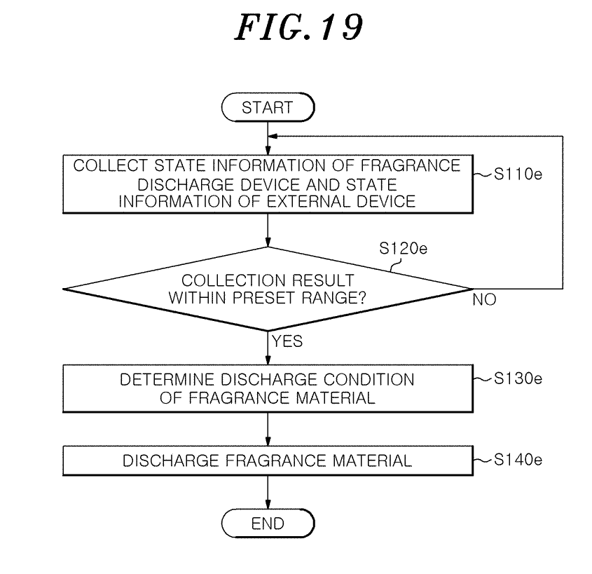

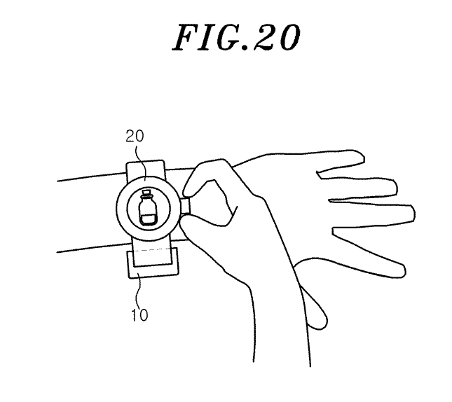

FIG. 19 is a flowchart illustrating a control method of a fragrance discharge device according to another embodiment, and FIG. 20 illustrates an example of applying the fragrance discharge device control method of FIG. 19.

Referring to FIGS. 19 and 20, the controller 110 of the fragrance discharge device 10 may discharge a fragrance material based on state information of the fragrance discharge device 10 or state information of the external device 20.

In detail, in operation S110e, the controller 110 may collect at least one of state information of the fragrance discharge device 10, such as an amount of time elapsed after wearing the fragrance discharge device 10, a remaining amount of the battery 130, an amount of the fragrance material remaining in the storage 160, a communication connection state with the external device 20, and a manipulation state of the user using the UI portion 150, and state information of the external device 20, such as an amount of time elapsed after wearing the external device 20, a remaining amount of the battery 230, a communication connection state, and a manipulation state of the user using the UI portion 250. For example, the controller 110 may receive and collect manipulation information of the user through the UI portion 250 of the external device 20.

In operation S120e, the controller 110 may determine whether the information collected in operation S110e is within the preset range. For example, the controller 110 may determine whether manipulation information of the UI portion 250 is a signal indicating discharging of the fragrance material.

When the collected information is determined to be included in the preset range, the controller 110 may determine a discharge condition for discharging the fragrance material in operation S130e and may control the discharge portion 170 and the driving portion/evaporator 180 to discharge the fragrance material to the outside based on the discharge condition in operation S140e. Here, the discharge condition may be adjusted to be different based on not only state information of the external device 20 but also state information of the fragrance discharge device 10, and different collection information. For example, although a signal indicating discharging of the fragrance material is received from the external device 20, the fragrance material may be set to not be discharged when a remaining fragrance material amount of the fragrance discharge device 10 is less than or equal to the preset range.

FIG. 20 illustrates an example in which the user inputs an instruction signal to discharge a fragrance material using a dial of a smart watch.

Although the present embodiment uses the manipulation information using the UI portion 250 of the external device 20 as an example, the present disclosure is not limited thereto. For example, the fragrance material may be set to be discharged when a preset period of time is elapsed after wearing the fragrance discharge device 10 or the external device 20.

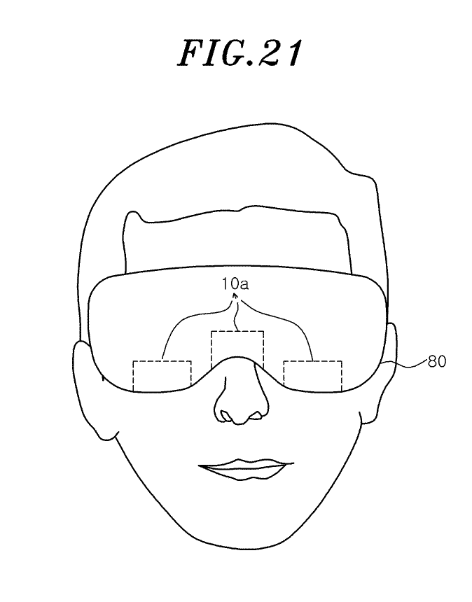

FIG. 21 illustrates an example of a fragrance discharge device according to another embodiment.

Referring to FIG. 21, one or more fragrance discharge devices 10a according to another embodiment may be installed in an HMD 80. Here, the HMD 80 may be a device for realizing VR or AR.

The HMD 80 may include a configuration corresponding to the aforementioned external device 20 and may interact with the fragrance discharge device 10a to control discharging of a fragrance material.

In detail, a controller of the HMD 80 may request the fragrance discharge device 10a to discharge a specific fragrance material based on content being played at the HMD 80. A plurality of fragrance materials corresponding to the content may be stored in the fragrance discharge device 10a and the fragrance discharge device 10a may discharge the specific fragrance material in response to the request of the HMD 80.

Also, when a plurality of discharge devices 10a is installed or when the fragrance discharge device 10a is capable of changing a discharge direction of a fragrance material, a direction in which the fragrance material is provided may be adjusted based on the content. Therefore, it is possible to further improve use satisfaction.

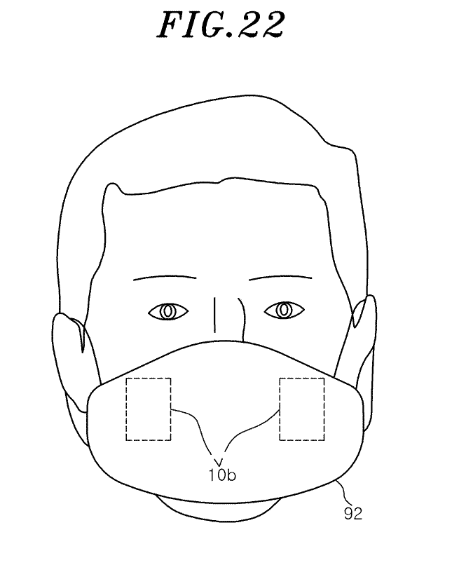

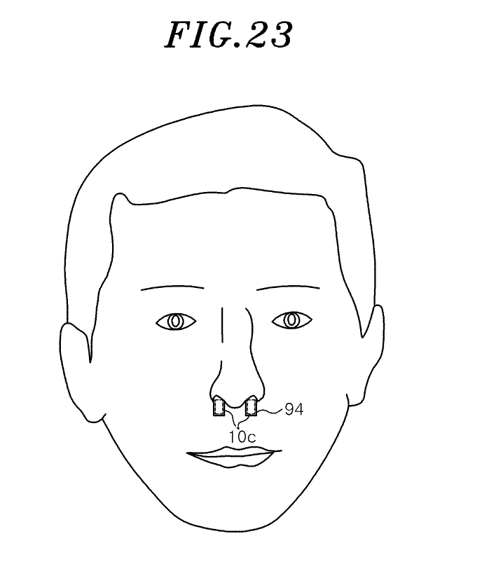

FIG. 22 illustrates an example of a fragrance discharge device according to another embodiment, and FIG. 23 illustrates an example of a fragrance discharge device according to another embodiment.

Referring to FIGS. 22 and 23, one or more fragrance discharge devices 10b according to another embodiment may be installed in a mask 92 in a form of covering a mouth and one or more fragrance discharge devices 10c according to another embodiment may be installed in a mask 94 in a form of inserting into a nose. Shapes and structures of a body of each of the discharge devices 10b and 10c and components thereof may be appropriately modified for installation purpose.

Here, each of the masks 92 and 94 may be configured to simply cover a respiratory organ and may include an intake filter for breathing and an exhaust filter, and may include a configuration corresponding to the aforementioned external device 20.

When the discharge devices 10b and 10c are directly provided to the masks 92 and 94, respectively, each configured to protect the respiratory organ, a fragrance material may be directly discharged toward the respiratory organ at a location adjacent thereto. Accordingly, effect of the fragrance material may be further improved.

A fragrance discharge device and a control method thereof according to the aforementioned embodiments may use a fragrance material suitable for a situation based on a variety of information associated with the user or the fragrance discharge device 10 and the external device 20, in addition to simply discharging the fragrance material. Therefore, usability, product utilization, and product satisfaction may be further enhanced. In particular, in the case of using context information associated with the user, such as motion information of the user, biometric information of the user, space information around the user, a time range set by the user, and a space range set by the user, a scenario for discharging a fragrance material may be configured to further meet the requirements of the user.

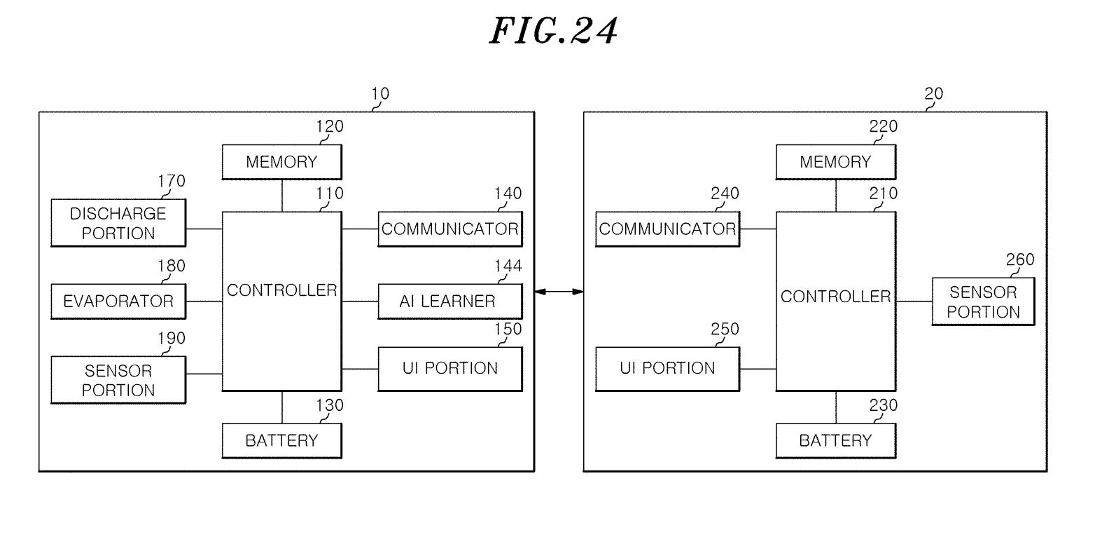

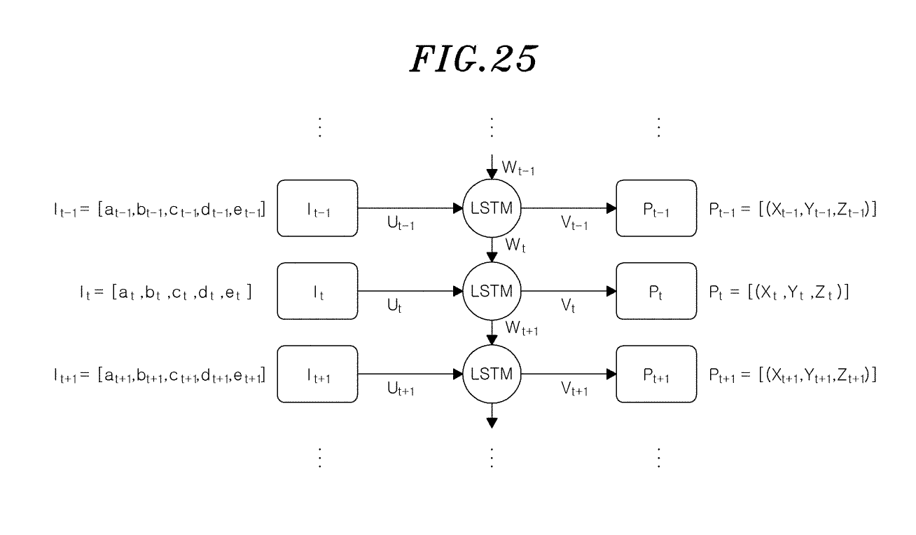

FIG. 24 is a block diagram illustrating a fragrance discharge device and an external device according to another embodiment, and FIG. 25 illustrates a learning process of an artificial intelligence (AI) learner of FIG. 24.

Referring to FIGS. 24 and 25, the fragrance discharge device 10 according to an embodiment may further include an AI learner 144 in addition to a configuration of the fragrance discharge device 10 shown in FIGS. 1 and 2.

The controller 110 may control the discharge portion 170 based on a result of detecting a motion of a user to selectively control discharging of a fragrance material. Also, the controller 110 may control the discharge portion 170 by further considering at least one of the aforementioned biometric information of the user, spatial information around the user, preset time span, preset space range, and state information of the fragrance discharge device 10.

The AI learner 144 may learn a control history of the controller 100. For example, the AI learner 144 may learn a control history on discharging of a fragrance material based on the biometric information of the user, the spatial information around the user, the time span, the space range, and the state information of the fragrance discharge device 10 while the user has been using the fragrance discharge device 10 from the past to the current time, and a change in the information. The control history may be, for example, a type, a discharge amount, a discharge direction, and a discharge pattern of the fragrance material. The AI learner 144 may construct the control history as a database.

The AI learner 144 may provide a learning result to the controller 110. Accordingly, the controller 110 may receive, from the AI learner 144, a recommendation on a control condition suitable for a situation in which the fragrance discharge device 10 operates to control discharging of the fragrance material. The AI learner 144 may learn the control history from the past to the current time using a time series data analysis method, for example, a recurrent neural network (RNN).

Also, the AI learner 144 may share the control history on discharging of the fragrance material with respect to a plurality of pieces of user information through a separate external server. The server may use the shared control history data for a time series data analysis.

The RNN may be suitable to learn a change in context over time in terms of AI learning. The RNN may analyze and learn an influence level of a primary factor based on state parameters of a previous time influencing over a current state. FIG. 25 illustrates the RNN. Here, an input vector (Iₜ) refers to context awareness based scent determination (CSD) information and may be the motion information of the user, the biometric information of the user, the spatial information around the user, the time span, the space range, and the state information of the discharge device 10. Also, an output vector (Pₜ) refers to a CSD value and may be a type, discharge concentration, and a discharge interval of the fragrance material. Also, U, W, and V refer to parameter vectors and may be a recursive method of learning a model by adjusting a weight of each parameter and using a result value acquired from the model as an input again. Each cell of the RNN may use a structure such as a long short-term memory (LSTM) and a gated recurrent unit (GRU).

Referring to FIG. 25, if I₀ is input, P₁ that is an output value (CSD) in a subsequent time is predicted. The subsequent prediction may be associated with state information of a previous time. Accordingly, P₂ is predicted using an input of I₁ and by adding information of I₀. That is, an RNN learning model has an accumulated value of the previous time and applies context of the previous time. Learning of the RNN is performed to decrease a difference between a predicted value and an actual input value. Here, U, W, and V may be determined using a method of minimizing an error between an actual value and a prediction value of the learning model.

The aforementioned description is provided as an example only and thus, one of ordinary skill in the art to which the disclosure pertains may understand that various modifications may be made without departing from the technical spirit or features of the disclosure. Therefore, the aforementioned embodiments should be understood as examples and construed as not be limiting.

The scope of the disclosure is defined by the claims and the equivalents and all of the changes or modifications from the scope of the claims and the equivalents should be understood to be included in the scope of the disclosure.

What is claimed is:

1. A fragrance discharge device comprising:
a body configured to be wearable by a user;
a discharge portion configured to discharge, to an outside of the body, at least a portion of fragrance material supplied from a storage; an
a controller comprising a processing device configured to control the discharge portion to discharge a fragrance material based on a result of a detected motion of the user, wherein the storage is coupled to the body and configured to store the fragrance material.

2. The fragrance discharge device of claim 1, wherein the controller is configured to control the discharge portion by further considering at least one of biometric information of the user, spatial information around the user, a preset time span, a preset space range, and state information of the fragrance discharge device.

3. The fragrance discharge device of claim 2, wherein at least one of the biometric information, the time information, and the space range is received from an external device.

4. The fragrance discharge device of claim 1, wherein the device is connected to a sensor portion coupled to the body and configured to detect the motion.

5. The fragrance discharge device of claim 1, further comprising:
a communicator configured to receive a detection result from an external device comprising a sensor portion configured to detect the motion.

6. The fragrance discharge device of claim 5, wherein the controller is configured to transmit at least one of a stored amount of the fragrance material and discharge information of the fragrance material to the external device through the communicator.

7. The fragrance discharge device of claim 1, wherein the storage comprises a plurality of storage spaces configured to independently store a plurality of fragrance materials each emitting a different fragrance, respectively, and
the controller is configured to control one or at least two of the plurality of fragrance materials to be discharged through the discharge portion.

8. A fragrance discharge device comprising:
a storage configured to store fragrance material connected to a body, a discharge portion, and a controller, wherein:
the body is configured to be wearable by a user;
the discharge portion is configured to discharge, to an outside of the body, at least a portion of the fragrance material supplied from the storage; and
the controller comprises a processing device configured to control the discharge portion to discharge a fragrance material based on a result of a detected motion of the user.

9. The fragrance discharge device of claim 8, wherein the controller is configured to control the discharge portion by further considering at least one of biometric information of the user, spatial information around the user, a preset time span, a preset space range, and state information of the fragrance discharge device.

10. The fragrance discharge device of claim 9, wherein at least one of the biometric information, the time information, and the space range is received from an external device.

11. The fragrance discharge device of claim 8, wherein the device is connected to a sensor portion coupled to the body and configured to detect the motion.

12. The fragrance discharge device of claim 8, further comprising:
a communicator configured to receive a detection result from an external device comprising a sensor portion configured to detect the motion.

13. The fragrance discharge device of claim 12, wherein the controller is configured to transmit at least one of a stored amount of the fragrance material and discharge information of the fragrance material to the external device through the communicator.

14. The fragrance discharge device of claim 8, wherein the storage comprises a plurality of storage spaces configured to independently store a plurality of fragrance materials each emitting a different fragrance, respectively, and
the controller is configured to control one or at least two of the plurality of fragrance materials to be discharged through the discharge portion.

15. A computer-implemented method of a fragrance discharge device configured to be wearable by a user and to selectively discharge a fragrance material to an outside using a controller comprising a processing device, the method comprising:
transferring a result of detecting a motion of the user to the controller;
determining, by the controller, at least one of a type, an amount, a discharge direction, and a discharge pattern of the fragrance material to be discharged based on the result of detecting the motion of the user; and
discharging the fragrance material stored in a storage provided to a body wearable by the user through a discharge portion provided to the body, based on a determination of the controller.

16. The method of claim 15, wherein the determining comprises determining, by the controller, at least one of the type, the amount, the discharge direction, and the discharge pattern of the fragrance material by further using at least one of biometric information of the user, spatial information around the user, a preset time span, a preset space range, and state information of the fragrance discharge device.

17. The method of claim 16, further comprising:
receiving at least one of the biometric information, the time span, and the space range from an external device.

18. The method of claim 15, further comprising:
detecting the motion of the user using a sensor portion provided to the body.

19. The method of claim 15, further comprising:
receiving a detection result from an external device comprising a sensor portion configured to detect the motion, through a communicator.

20. The method of claim 19, further comprising:
transmitting at least one of a stored amount of the fragrance material and discharge information of the fragrance material to the external device through the communicator.
